(19) United States
(12) Patent Application Publication
(10) Pub. No.: US 2026/0338680 A1
(43) Pub. Date: Oct. 29, 2026

(54) CARBONATES CHARACTERIZATION VIA DIFFUSE REFLECTION INFRARED MEASUREMENT

(71) Applicant: SCHLUMBERGER TECHNOLOGY CORPORATION, Sugar Land, TX (US)

(72) Inventors: Mahdi AMMAR, Dhahran (SA); Alexis BARTHET, Fontainebleau (FR)

(21) Appl. No.: 19/114,475

(22) PCT Filed: Sep. 22, 2023
(86) PCT No.: PCT/US2023/033431

§ 371 Date: Mar. 24, 2025

(30) Foreign Application Priority Data
Sep. 22, 2022 (EP) 22306394.2

(51) Int. Cl. G01N 21/3563; G01N 21/47; G01N 33/24

(52) U.S. Cl. CPC G01N 21/3563; G01N 21/4738; G01N 33/241

(57) ABSTRACT

A method can include irradiating a rock sample with infrared radiation from at least one radiation source; detecting infrared radiation reflected from the rock sample for two different wavelength bands using a photodetector; and, based on a comparison of the infrared radiation for the two different wavelength bands, using a processor, determining whether the rock sample includes carbonate.

### CROSS-REFERENCE TO RELATED APPLICATIONS

This application claims priority to European Patent Application No. 22306394.2, which was filed on Sep. 22, 2022, and is incorporated herein by reference in its entirety.

### BACKGROUND

A reservoir can be a subsurface formation that can be characterized at least in part by its porosity and fluid permeability. As an example, a reservoir may be part of a basin such as a sedimentary basin. A basin can be a depression (e.g., caused by plate tectonic activity, subsidence, etc.) in which sediments accumulate. As an example, where hydrocarbon source rocks occur in combination with appropriate depth and duration of burial, a petroleum system may develop within a basin, which may form a reservoir that includes hydrocarbon fluids (e.g., oil, gas, etc.). Various operations may be performed in the field to access such hydrocarbon fluids and/or produce such hydrocarbon fluids. For example, consider equipment operations where equipment may be controlled to perform one or more operations. In such an example, control may be based at least in part on characteristics of rock, which can be present as three-dimensional objects in drilling fluid (e.g., mud) such as, for example, rock cuttings that may be generated by a drill bit breaking rock.

### SUMMARY

A method can include irradiating a rock sample with infrared radiation from at least one radiation source; detecting infrared radiation reflected from the rock sample for two different wavelength bands using a photodetector; and, based on a comparison of the infrared radiation for the two different wavelength bands, using a processor, determining whether the rock sample includes carbonate.

A system can include a processor; memory accessible to the processor; and processor-executable instructions stored in the memory to instruct the system to: irradiate a rock sample with infrared radiation from at least one radiation source; detect infrared radiation reflected from the rock sample for two different wavelength bands using a photodetector; and based on a comparison of the infrared radiation for the two different wavelength bands, determine whether the rock sample comprises carbonate.

One or more non-transitory computer-readable storage media can include processor-executable instructions to instruct a computing system to: irradiate a rock sample with infrared radiation from at least one radiation source; detect infrared radiation reflected from the rock sample for two different wavelength bands using a photodetector; and, based on a comparison of the infrared radiation for the two different wavelength bands, determine whether the rock sample comprises carbonate.

This summary is provided to introduce a selection of concepts that are further described below in the detailed description. This summary is not intended to identify key or essential features of the claimed subject matter, nor is it intended to be used as an aid in limiting the scope of the claimed subject matter.

### BRIEF DESCRIPTION OF THE DRAWINGS

Features and advantages of the described implementations can be more readily understood by reference to the following description taken in conjunction with the accompanying drawings.

FIG. 1 illustrates an example system that includes various framework components associated with one or more geologic environments;

FIG. 2 illustrates an example of a system;

FIG. 3 illustrates an example of a drilling equipment and examples of borehole shapes;

FIG. 4 illustrates an example of a system and an example of a spectrum;

FIG. 5 illustrates examples of spectra;

FIG. 6 illustrates an example of a plot;

FIG. 7 illustrates an example of a system;

FIG. 8 illustrates an example of a system;

FIG. 9 illustrates an example of a plot;

FIG. 10 illustrates examples of systems;

FIG. 11 illustrates an example of a detector array;

FIG. 12 illustrates an example of a plot;

FIG. 13 illustrates an example of a plot;

FIG. 14 illustrates an example of a plot;

FIG. 15 illustrates an example of a system;

FIG. 16 illustrates an example of a plot;

FIG. 17 illustrates an example of a method and an example of a system;

FIG. 18 illustrates examples of computer and network equipment; and

FIG. 19 illustrates example components of a system and a networked system.

### DETAILED DESCRIPTION

This description is not to be taken in a limiting sense, but rather is made merely for the purpose of describing the general principles of the implementations. The scope of the described implementations should be ascertained with reference to the issued claims.

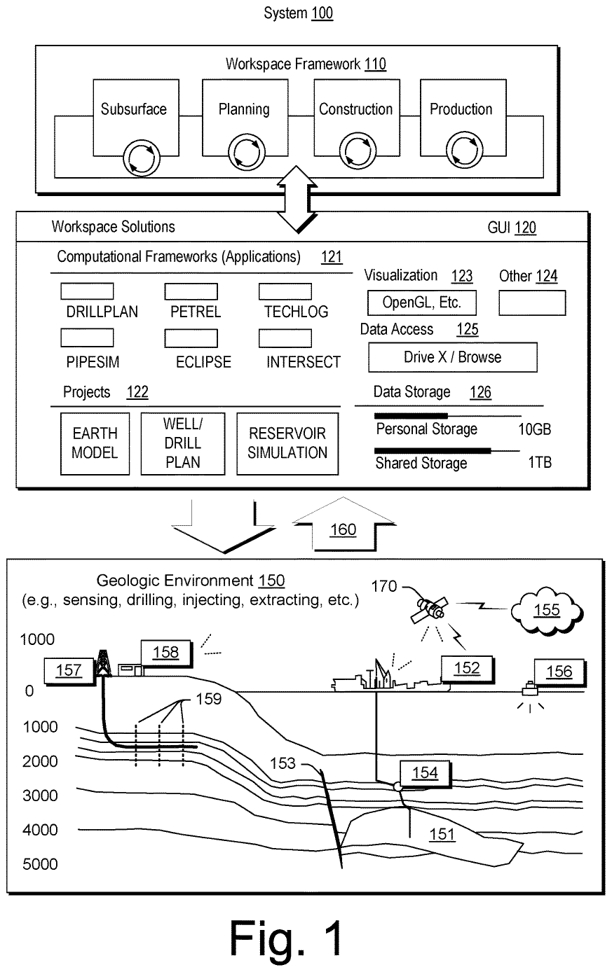

FIG. 1 shows an example of a system 100 that includes a workspace framework 110 that can provide for instantiation of, rendering of, interactions with, etc., a graphical user interface (GUI) 120. In the example of FIG. 1, the GUI 120 can include graphical controls for computational frameworks (e.g., applications) 121, projects 122, visualization 123, one or more other features 124, data access 125, and data storage 126.

In the example of FIG. 1, the workspace framework 110 may be tailored to a particular geologic environment such as an example geologic environment 150. For example, the geologic environment 150 may include layers (e.g., stratification) that include a reservoir 151 and that may be intersected by a fault 153. A geologic environment 150 may be outfitted with a variety of sensors, detectors, actuators, etc. In such an environment, various types of equipment such as, for example, equipment 152 may include communication circuitry to receive and to transmit information, optionally with respect to one or more networks 155. Such information may include information associated with downhole equipment 154, which may be equipment to acquire information, to assist with resource recovery, etc. Other equipment 156 may be located remote from a wellsite and include sensing, detecting, emitting, or other circuitry. Such equipment may include storage and communication circuitry to store and to communicate data, instructions, etc. One or more satellites may be provided for purposes of communications, data acquisition, etc. For example, FIG. 1 shows a satellite 170 in communication with the network 155 that may be configured for communications, noting that the satellite may additionally or alternatively include circuitry for imagery (e.g., spatial, spectral, temporal, radiometric, etc.).

FIG. 1 also shows the geologic environment 150 as optionally including equipment 157 and 158 associated with a well that includes a substantially horizontal portion that may intersect with one or more fractures 159. For example, consider a well in a formation that may include natural fractures, artificial fractures (e.g., hydraulic fractures) or a combination of natural and artificial fractures. As an example, a well may be drilled for a reservoir that is laterally extensive. In such an example, lateral variations in properties, stresses, etc., may exist where an assessment of such variations may assist with planning, operations, etc., to develop a laterally extensive reservoir (e.g., via fracturing, injecting, extracting, etc.). As an example, the equipment 157 and/or 158 may include components, a system, systems, etc. for fracturing, seismic sensing, analysis of seismic data, assessment of one or more fractures, etc.

In the example of FIG. 1, the GUI 120 shows some examples of computational frameworks, including the DRILLPLAN, PETREL, TECHLOG, PETROMOD, ECLIPSE, and INTERSECT and DRILLOPS frameworks (SLB, Houston, Texas), noting that one or more other frameworks, additionally or alternatively, may be included or otherwise accessible.

The DRILLPLAN framework provides for digital well construction planning and includes features for automation of repetitive tasks and validation workflows, enabling improved quality drilling programs (e.g., digital drilling plans, etc.) to be produced quickly with assured coherency. The DRILLOPS framework provides for execution of a digital drilling plan and can facility plan adherence, while delivering goal-based automation. Automation may utilize data analysis and learning systems to assist and optimize tasks, such as, for example, setting ROP to drilling a stand. Well construction activities (e.g., tripping, drilling, cementing, etc.) may be continually monitored and dynamically updated using feedback from operational activities. The DRILLOPS framework may provide for various levels of automation based on planning and/or re-planning (e.g., via the DRILLPLAN framework), feedback, etc.

The PETREL framework can be part of the DELFI cognitive exploration and production (E&P) environment (SLB, Houston, Texas, referred to as the DELFI environment) for utilization in geosciences and geoengineering, for example, to analyze subsurface data from exploration to production of fluid from a reservoir.

One or more types of frameworks may be implemented within or in a manner operatively coupled to the DELFI environment, which is a secure, cognitive, cloud-based collaborative environment that integrates data and workflows with digital technologies, such as artificial intelligence (AI) and machine learning (ML). Such an environment can provide for operations that involve one or more frameworks. The DELFI environment may be referred to as the DELFI framework, which may be a framework of frameworks. The DELFI environment can include various other frameworks, which may operate using one or more types of models (e.g., simulation models, etc.).

The TECHLOG framework can handle and process field and laboratory data for a variety of geologic environments (e.g., deepwater exploration, shale, etc.). The TECHLOG framework can structure wellbore data for analyses, planning, etc.

The PIPESIM simulator includes solvers that may provide simulation results such as, for example, multiphase flow results (e.g., from a reservoir to a wellhead and beyond, etc.), flowline and surface facility performance, etc. The PIPESIM simulator may be integrated, for example, with the AVOCET production operations framework (SLB, Houston Texas). The PIPESIM simulator may be an optimizer that can optimize one or more operational scenarios at least in part via simulation of physical phenomena.

The ECLIPSE framework provides a reservoir simulator with numerical solvers for prediction of dynamic behavior for various types of reservoirs and development schemes.

The INTERSECT framework provides a high-resolution reservoir simulator for simulation of geological features and quantification of uncertainties, for example, by creating production scenarios and, with the integration of precise models of the surface facilities and field operations, the INTERSECT framework can produce results, which may be continuously updated by real-time data exchanges (e.g., from one or more types of data acquisition equipment in the field that can acquire data during one or more types of field operations, etc.). The INTERSECT framework can provide completion configurations for complex wells where such configurations can be built in the field, can provide detailed chemical-enhanced-oil-recovery (EOR) formulations where such formulations can be implemented in the field, can analyze application of steam injection and other thermal EOR techniques for implementation in the field, advanced production controls in terms of reservoir coupling and flexible field management, and flexibility to script customized solutions for improved modeling and field management control. The INTERSECT framework, as with the other example frameworks, may be utilized as part of the DELFI environment, for example, for rapid simulation of multiple concurrent cases.

The aforementioned DELFI environment provides various features for workflows as to subsurface analysis, planning, construction and production, for example, as illustrated in the workspace framework 110. As shown in FIG. 1, outputs from the workspace framework 110 can be utilized for directing, controlling, etc., one or more processes in the geologic environment 150, and feedback 160 can be received via one or more interfaces in one or more forms (e.g., acquired data as to operational conditions, equipment conditions, environment conditions, etc.).

In the example of FIG. 1, the visualization features 123 may be implemented via the workspace framework 110, for example, to perform tasks as associated with one or more of subsurface regions, planning operations, constructing wells and/or surface fluid networks, and producing from a reservoir.

Visualization features may provide for visualization of various earth models, properties, etc., in one or more dimensions. As an example, visualization features may include one or more control features for control of equipment, which can include, for example, field equipment that can perform one or more field operations. A workflow may utilize one or more frameworks to generate information that can be utilized to control one or more types of field equipment (e.g., drilling equipment, wireline equipment, fracturing equipment, etc.).

As to a reservoir model that may be suitable for utilization by a simulator, consider acquisition of seismic data as acquired via reflection seismology, which finds use in geophysics, for example, to estimate properties of subsurface formations. Seismic data may be processed and interpreted, for example, to understand better composition, fluid content, extent and geometry of subsurface rocks. Such interpretation results can be utilized to plan, simulate, perform, etc., one or more operations for production of fluid from a reservoir (e.g., reservoir rock, etc.). Field acquisition equipment may be utilized to acquire seismic data, which may be in the form of traces where a trace can include values organized with respect to time and/or depth (e.g., consider 1D, 2D, 3D or 4D seismic data).

A model may be a simulated version of a geologic environment where a simulator may include features for simulating physical phenomena in a geologic environment based at least in part on a model or models. A simulator, such as a reservoir simulator, can simulate fluid flow in a geologic environment based at least in part on a model that can be generated via a framework that receives seismic data. A simulator can be a computerized system (e.g., a computing system) that can execute instructions using one or more processors to solve a system of equations that describe physical phenomena subject to various constraints. In such an example, the system of equations may be spatially defined (e.g., numerically discretized) according to a spatial model that that includes layers of rock, geobodies, etc., that have corresponding positions that can be based on interpretation of seismic and/or other data. A spatial model may be a cell-based model where cells are defined by a grid (e.g., a mesh). A cell in a cell-based model can represent a physical area or volume in a geologic environment where the cell can be assigned physical properties (e.g., permeability, fluid properties, etc.) that may be germane to one or more physical phenomena (e.g., fluid volume, fluid flow, pressure, etc.). A reservoir simulation model can be a spatial model that may be cell-based.

While several simulators are illustrated in the example of FIG. 1, one or more other simulators may be utilized, additionally or alternatively. For example, consider the VISAGE geomechanics simulator SLB, Houston Texas) or the PETROMOD simulator (SLB, Houston Texas), etc. The VISAGE simulator includes finite element numerical solvers that may provide simulation results such as, for example, results as to compaction and subsidence of a geologic environment, well and completion integrity in a geologic environment, cap-rock and fault-seal integrity in a geologic environment, fracture behavior in a geologic environment, thermal recovery in a geologic environment, CO₂ disposal, etc. The PETROMOD framework provides petroleum systems modeling capabilities that can combine one or more of seismic, well, and geological information to model the evolution of a sedimentary basin. The PETROMOD framework can predict if, and how, a reservoir has been charged with hydrocarbons, including the source and timing of hydrocarbon generation, migration routes, quantities, and hydrocarbon type in the subsurface or at surface conditions. The MANGROVE simulator (SLB, Houston, Texas) provides for optimization of stimulation design (e.g., stimulation treatment operations such as hydraulic fracturing) in a reservoir-centric environment. The MANGROVE framework can combine scientific and experimental work to predict geomechanical propagation of hydraulic fractures, reactivation of natural fractures, etc., along with production forecasts within 3D reservoir models (e.g., production from a drainage area of a reservoir where fluid moves via one or more types of fractures to a well and/or from a well). The MANGROVE framework can provide results pertaining to heterogeneous interactions between hydraulic and natural fracture networks, which may assist with optimization of the number and location of fracture treatment stages (e.g., stimulation treatment(s)), for example, to increased perforation efficiency and recovery.

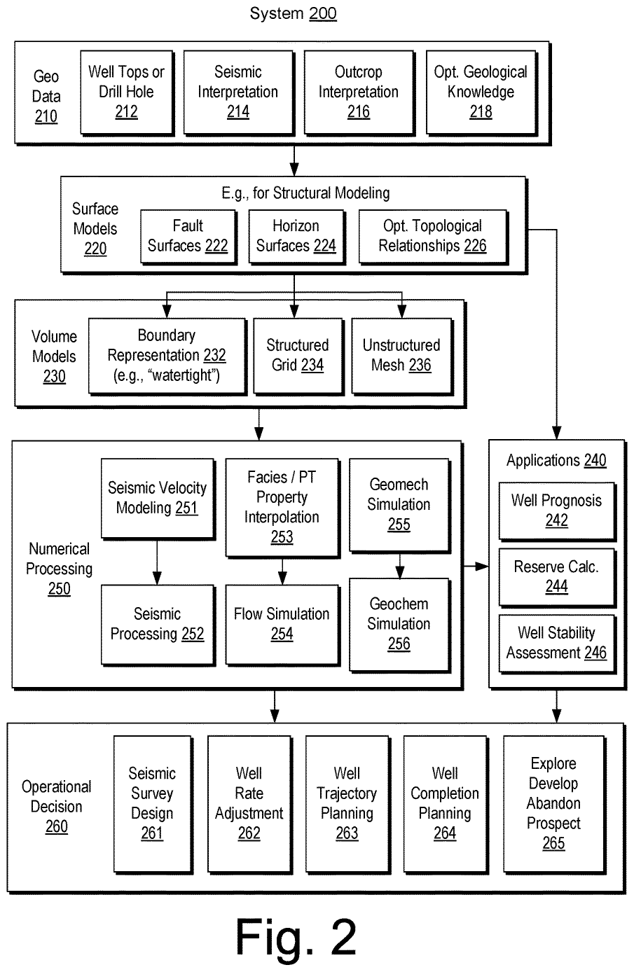

FIG. 2 shows an example of a system 200 that can be operatively coupled to one or more databases, data streams, etc. For example, one or more pieces of field equipment, laboratory equipment, computing equipment (e.g., local and/or remote), etc., can provide and/or generate data that may be utilized in the system 200.

As shown, the system 200 can include a geological/geophysical data block 210, a surface models block 220 (e.g., for one or more structural models), a volume modules block 230, an applications block 240, a numerical processing block 250 and an operational decision block 260. As shown in the example of FIG. 2, the geological/geophysical data block 210 can include data from well tops or drill holes 212, data from seismic interpretation 214, data from outcrop interpretation and optionally data from geological knowledge. As an example, the geological/geophysical data block 210 can include data from digital images, which can include digital images of cores, cuttings, cavings, outcrops, etc. As to the surface models block 220, it may provide for creation, editing, etc. of one or more surface models based on, for example, one or more of fault surfaces 222, horizon surfaces 224 and optionally topological relationships 226. As to the volume models block 230, it may provide for creation, editing, etc. of one or more volume models based on, for example, one or more of boundary representations 232 (e.g., to form a watertight model), structured grids 234 and unstructured meshes 236.

As shown in the example of FIG. 2, the system 200 may allow for implementing one or more workflows, for example, where data of the data block 210 are used to create, edit, etc. one or more surface models of the surface models block 220, which may be used to create, edit, etc. one or more volume models of the volume models block 230. As indicated in the example of FIG. 2, the surface models block 220 may provide one or more structural models, which may be input to the applications block 240. For example, such a structural model may be provided to one or more applications, optionally without performing one or more processes of the volume models block 230 (e.g., for purposes of numerical processing by the numerical processing block 250). Accordingly, the system 200 may be suitable for one or more workflows for structural modeling (e.g., optionally without performing numerical processing per the numerical processing block 250).

As to the applications block 240, it may include applications such as a well prognosis application 242, a reserve calculation application 244 and a well stability assessment application 246. As to the numerical processing block 250, it may include a process for seismic velocity modeling 251 followed by seismic processing 252, a process for facies and petrophysical property interpolation 253 followed by flow simulation 254, and a process for geomechanical simulation 255 followed by geochemical simulation 256. As indicated, as an example, a workflow may proceed from the volume models block 230 to the numerical processing block 250 and then to the applications block 240 and/or to the operational decision block 260. As another example, a workflow may proceed from the surface models block 220 to the applications block 240 and then to the operational decisions block 260 (e.g., consider an application that operates using a structural model).

In the example of FIG. 2, the operational decisions block 260 may include a seismic survey design process 261, a well rate adjustment process 252, a well trajectory planning process 263, a well completion planning process 264 and a process for one or more prospects, for example, to decide whether to explore, develop, abandon, etc. a prospect.

Referring again to the data block 210, the well tops or drill hole data 212 may include spatial localization, and optionally surface dip, of an interface between two geological formations or of a subsurface discontinuity such as a geological fault; the seismic interpretation data 214 may include a set of points, lines or surface patches interpreted from seismic reflection data, and representing interfaces between media (e.g., geological formations in which seismic wave velocity differs) or subsurface discontinuities; the outcrop interpretation data 216 may include a set of lines or points, optionally associated with measured dip, representing boundaries between geological formations or geological faults, as interpreted on the earth surface; and the geological knowledge data 218 may include, for example knowledge of the paleo-tectonic and sedimentary evolution of a region.

As to a structural model, it may be, for example, a set of gridded or meshed surfaces representing one or more interfaces between geological formations (e.g., horizon surfaces) or mechanical discontinuities (fault surfaces) in the subsurface. As an example, a structural model may include some information about one or more topological relationships between surfaces (e.g. fault A truncates fault B, fault B intersects fault C, etc.).

As to the one or more boundary representations 232, they may include a numerical representation in which a subsurface model is partitioned into various closed units representing geological layers and fault blocks where an individual unit may be defined by its boundary and, optionally, by a set of internal boundaries such as fault surfaces.

As to the one or more structured grids 234, it may include a grid that partitions a volume of interest into different elementary volumes (cells), for example, that may be indexed according to a pre-defined, repeating pattern. As to the one or more unstructured meshes 236, it may include a mesh that partitions a volume of interest into different elementary volumes, for example, that may not be readily indexed following a pre-defined, repeating pattern (e.g., consider a Cartesian cube with indexes I, J, and K, along x, y, and z axes).

As to the seismic velocity modeling 251, it may include calculation of velocity of propagation of seismic waves (e.g., where seismic velocity depends on type of seismic wave and on direction of propagation of the wave). As to the seismic processing 252, it may include a set of processes allowing identification of localization of seismic reflectors in space, physical characteristics of the rocks in between these reflectors, etc.

As to the facies and petrophysical property interpolation 253, it may include an assessment of type of rocks and of their petrophysical properties (e.g., porosity, permeability), for example, optionally in areas not sampled by well logs or coring. As an example, such an interpolation may be constrained by interpretations from log and core data, and by prior geological knowledge.

As to the flow simulation 254, as an example, it may include simulation of flow of hydro-carbons in the subsurface, for example, through geological times (e.g., in the context of petroleum systems modeling, when trying to predict the presence and quality of oil in an un-drilled formation) or during the exploitation of a hydrocarbon reservoir (e.g., when some fluids are pumped from or into the reservoir).

As to geomechanical simulation 255, it may include simulation of the deformation of rocks under boundary conditions. Such a simulation may be used, for example, to assess compaction of a reservoir (e.g., associated with its depletion, when hydrocarbons are pumped from the porous and deformable rock that composes the reservoir). As an example, a geomechanical simulation may be used for a variety of purposes such as, for example, prediction of fracturing, reconstruction of the paleo-geometries of the reservoir as they were prior to tectonic deformations, etc.

As to geochemical simulation 256, such a simulation may simulate evolution of hydrocarbon formation and composition through geological history (e.g., to assess the likelihood of oil accumulation in a particular subterranean formation while exploring new prospects).

As to the various applications of the applications block 240, the well prognosis application 242 may include predicting type and characteristics of geological formations that may be encountered by a drill bit, and location where such rocks may be encountered (e.g., before a well is drilled); the reserve calculations application 244 may include assessing total amount of hydrocarbons or ore material present in a subsurface environment (e.g., and estimates of which proportion can be recovered, given a set of economic and technical constraints); and the well stability assessment application 246 may include estimating risk that a well, already drilled or to-be-drilled, will collapse or be damaged due underground stress.

As to the operational decision block 260, the seismic survey design process 261 may include deciding where to place seismic sources and receivers to optimize the coverage and quality of the collected seismic information while minimizing cost of acquisition; the well rate adjustment process 262 may include controlling injection and production well schedules and rates (e.g., to maximize recovery and production); the well trajectory planning process 263 may include designing a well trajectory to maximize potential recovery and production while minimizing drilling risks and costs; the well trajectory planning process 264 may include selecting proper well tubing, casing and completion (e.g., to meet expected production or injection targets in specified reservoir formations); and the prospect process 265 may include decision making, in an exploration context, to continue exploring, start producing or abandon prospects (e.g., based on an integrated assessment of technical and financial risks against expected benefits).

The system 200 can include and/or can be operatively coupled to a system such as the system 100 of FIG. 1. For example, the workspace framework 110 may provide for instantiation of, rendering of, interactions with, etc., the graphical user interface (GUI) 120 to perform one or more actions as to the system 200. In such an example, access may be provided to one or more frameworks (e.g., DRILLPLAN, PETREL, TECHLOG, PIPESIM, ECLIPSE, INTERSECT, DRILLOPS, etc.). One or more frameworks may provide for geo data acquisition as in block 210, for structural modeling as in block 220, for volume modeling as in block 230, for running an application as in block 240, for numerical processing as in block 250, for operational decision making as in block 260, etc.

As an example, the system 200 may provide for monitoring data, which can include geo data per the geo data block 210. In various examples, geo data may be acquired during one or more operations. For example, consider acquiring geo data during drilling operations via downhole equipment and/or surface equipment. As an example, the operational decision block 260 can include capabilities for monitoring, analyzing, etc., such data for purposes of making one or more operational decisions, which may include controlling equipment, revising operations, revising a plan, etc. In such an example, data may be fed into the system 200 at one or more points where the quality of the data may be of particular interest. For example, data quality may be characterized by one or more metrics where data quality may provide indications as to trust, probabilities, etc., which may be germane to operational decision making and/or other decision making.

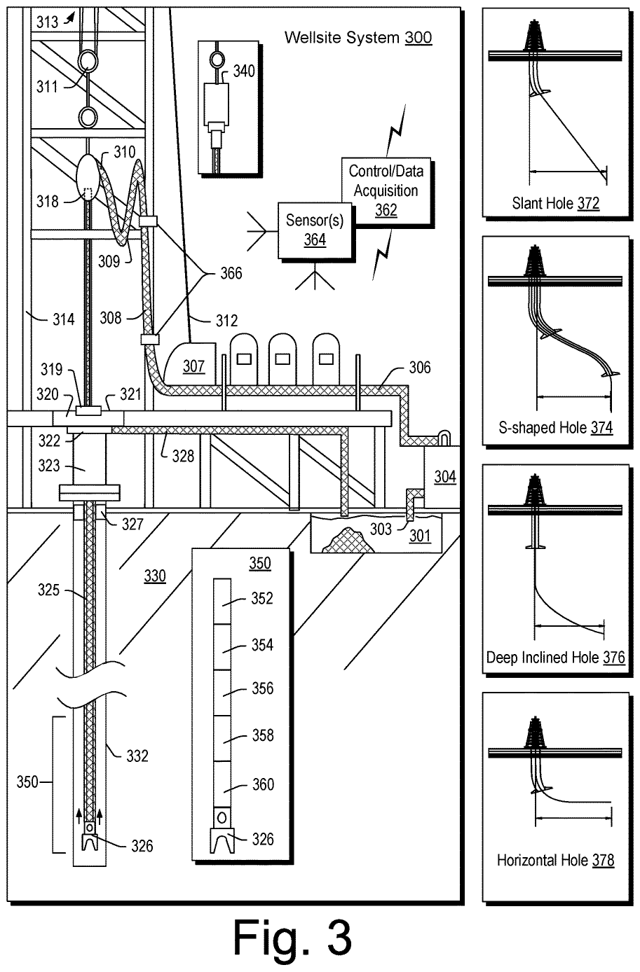

FIG. 3 shows an example of a wellsite system 300 (e.g., at a wellsite that may be onshore or offshore). As shown, the wellsite system 300 can include a mud tank 301 for holding mud and other material (e.g., where mud can be a drilling fluid), a suction line 303 that serves as an inlet to a mud pump 304 for pumping mud from the mud tank 301 such that mud flows to a vibrating hose 306, a drawworks 307 for winching drill line or drill lines 312, a standpipe 308 that receives mud from the vibrating hose 306, a kelly hose 309 that receives mud from the standpipe 308, a gooseneck or goosenecks 310, a traveling block 311, a crown block 313 for carrying the traveling block 311 via the drill line or drill lines 312, a derrick 314, a kelly 318 or a top drive 340, a kelly drive bushing 319, a rotary table 320, a drill floor 321, a bell nipple 322, one or more blowout preventors (BOPs) 323, a drillstring 325, a drill bit 326, a casing head 327 and a flow pipe 328 that carries mud and other material to, for example, the mud tank 301. As an example, rock cuttings may be carried by drilling fluid and deposited in the mud tank 301 and/or removed prior to return of drilling fluid to the mud tank 301.

In the example system of FIG. 3, a borehole 332 is formed in subsurface formations 330 by rotary drilling; noting that various example embodiments may also use one or more directional drilling techniques, equipment, etc.

As shown in the example of FIG. 3, the drillstring 325 is suspended within the borehole 332 and has a drillstring assembly 350 that includes the drill bit 326 at its lower end. As an example, the drillstring assembly 350 may be a bottom hole assembly (BHA).

The wellsite system 300 can provide for operation of the drillstring 325 and other operations. As shown, the wellsite system 300 includes the traveling block 311 and the derrick 314 positioned over the borehole 332. As mentioned, the wellsite system 300 can include the rotary table 320 where the drillstring 325 pass through an opening in the rotary table 320.

As shown in the example of FIG. 3, the wellsite system 300 can include the kelly 318 and associated components, etc., or the top drive 340 and associated components. As to a kelly example, the kelly 318 may be a square or hexagonal metal/alloy bar with a hole drilled therein that serves as a mud flow path. The kelly 318 can be used to transmit rotary motion from the rotary table 320 via the kelly drive bushing 319 to the drillstring 325, while allowing the drillstring 325 to be lowered or raised during rotation. The kelly 318 can pass through the kelly drive bushing 319, which can be driven by the rotary table 320. As an example, the rotary table 320 can include a master bushing that operatively couples to the kelly drive bushing 319 such that rotation of the rotary table 320 can turn the kelly drive bushing 319 and hence the kelly 318. The kelly drive bushing 319 can include an inside profile matching an outside profile (e.g., square, hexagonal, etc.) of the kelly 318; however, with slightly larger dimensions so that the kelly 318 can freely move up and down inside the kelly drive bushing 319.

As to a top drive example, the top drive 340 can provide functions performed by a kelly and a rotary table. The top drive 340 can turn the drillstring 325. As an example, the top drive 340 can include one or more motors (e.g., electric and/or hydraulic) connected with appropriate gearing to a short section of pipe called a quill, that in turn may be screwed into a saver sub or the drillstring 325 itself. The top drive 340 can be suspended from the traveling block 311, so the rotary mechanism is free to travel up and down the derrick 314. As an example, a top drive 340 may allow for drilling to be performed with more joint stands than a kelly/rotary table approach.

In the example of FIG. 3, the mud tank 301 can hold mud, which can be one or more types of drilling fluids. As an example, a wellbore may be drilled to produce fluid, inject fluid or both (e.g., hydrocarbons, minerals, water, etc.).

In the example of FIG. 3, the drillstring 325 (e.g., including one or more downhole tools) may be composed of a series of pipes threadably connected together to form a long tube with the drill bit 326 at the lower end thereof. As the drillstring 325 is advanced into a wellbore for drilling, at some point in time prior to or coincident with drilling, the mud may be pumped by the pump 304 from the mud tank 301 (e.g., or other source) via the lines 306, 308 and 309 to a port of the kelly 318 or, for example, to a port of the top drive 340. The mud can then flow via a passage (e.g., or passages) in the drillstring 325 and out of ports located on the drill bit 326 (see, e.g., a directional arrow). As the mud exits the drillstring 325 via ports in the drill bit 326, it can then circulate upwardly through an annular region between an outer surface(s) of the drillstring 325 and surrounding wall(s) (e.g., open borehole, casing, etc.), as indicated by directional arrows. In such a manner, the mud lubricates the drill bit 326 and carries heat energy (e.g., frictional or other energy) and formation cuttings to the surface where the mud may be returned to the mud tank 301, for example, for recirculation with processing to remove cuttings and other material.

In the example of FIG. 3, processed mud pumped by the pump 304 into the drillstring 325 may, after exiting the drillstring 325, form a mudcake that lines the wellbore which, among other functions, may reduce friction between the drillstring 325 and surrounding wall(s) (e.g., borehole, casing, etc.). A reduction in friction may facilitate advancing or retracting the drillstring 325. During a drilling operation, the entire drillstring 325 may be pulled from a wellbore and optionally replaced, for example, with a new or sharpened drill bit, a smaller diameter drillstring, etc. As mentioned, the act of pulling a drillstring out of a hole or replacing it in a hole is referred to as tripping. A trip may be referred to as an upward trip or an outward trip or as a downward trip or an inward trip depending on trip direction.

As an example, consider a downward trip where upon arrival of the drill bit 326 of the drillstring 325 at a bottom of a wellbore, pumping of the mud commences to lubricate the drill bit 326 for purposes of drilling to enlarge the wellbore. As mentioned, the mud can be pumped by the pump 304 into a passage of the drillstring 325 and, upon filling of the passage, the mud may be used as a transmission medium to transmit energy, for example, energy that may encode information as in mud-pulse telemetry. Characteristics of the mud can be utilized to determine how pulses are transmitted (e.g., pulse shape, energy loss, transmission time, etc.).

As an example, mud-pulse telemetry equipment may include a downhole device configured to effect changes in pressure in the mud to create an acoustic wave or waves upon which information may modulated. In such an example, information from downhole equipment (e.g., one or more modules of the drillstring 325) may be transmitted uphole to an uphole device, which may relay such information to other equipment for processing, control, etc.

As an example, telemetry equipment may operate via transmission of energy via the drillstring 325 itself. For example, consider a signal generator that imparts coded energy signals to the drillstring 325 and repeaters that may receive such energy and repeat it to further transmit the coded energy signals (e.g., information, etc.).

As an example, the drillstring 325 may be fitted with telemetry equipment 352 that includes a rotatable drive shaft, a turbine impeller mechanically coupled to the drive shaft such that the mud can cause the turbine impeller to rotate, a modulator rotor mechanically coupled to the drive shaft such that rotation of the turbine impeller causes said modulator rotor to rotate, a modulator stator mounted adjacent to or proximate to the modulator rotor such that rotation of the modulator rotor relative to the modulator stator creates pressure pulses in the mud, and a controllable brake for selectively braking rotation of the modulator rotor to modulate pressure pulses. In such example, an alternator may be coupled to the aforementioned drive shaft where the alternator includes at least one stator winding electrically coupled to a control circuit to selectively short the at least one stator winding to electromagnetically brake the alternator and thereby selectively brake rotation of the modulator rotor to modulate the pressure pulses in the mud.

In the example of FIG. 3, an uphole control and/or data acquisition system 362 may include circuitry to sense pressure pulses generated by telemetry equipment 352 and, for example, communicate sensed pressure pulses or information derived therefrom for process, control, etc.

The assembly 350 of the illustrated example includes a logging-while-drilling (LWD) module 354, a measurement-while-drilling (MWD) module 356, an optional module 358, a rotary-steerable system (RSS) and/or motor 360, and the drill bit 326. Such components or modules may be referred to as tools where a drillstring can include a plurality of tools.

As to a RSS, it involves technology utilized for directional drilling. Directional drilling involves drilling into the Earth to form a deviated bore such that the trajectory of the bore is not vertical; rather, the trajectory deviates from vertical along one or more portions of the bore. As an example, consider a target that is located at a lateral distance from a surface location where a rig may be stationed. In such an example, drilling can commence with a vertical portion and then deviate from vertical such that the bore is aimed at the target and, eventually, reaches the target. Directional drilling may be implemented where a target may be inaccessible from a vertical location at the surface of the Earth, where material exists in the Earth that may impede drilling or otherwise be detrimental (e.g., consider a salt dome, etc.), where a formation is laterally extensive (e.g., consider a relatively thin yet laterally extensive reservoir), where multiple bores are to be drilled from a single surface bore, where a relief well is desired, etc.

One approach to directional drilling involves a mud motor; however, a mud motor can present some challenges depending on factors such as rate of penetration (ROP), transferring weight to a bit (e.g., weight on bit, WOB) due to friction, etc. A mud motor can be a positive displacement motor (PDM) that operates to drive a bit (e.g., during directional drilling, etc.). A PDM operates as drilling fluid is pumped through it where the PDM converts hydraulic power of the drilling fluid into mechanical power to cause the bit to rotate.

As an example, a PDM may operate in a combined rotating mode where surface equipment is utilized to rotate a bit of a drillstring (e.g., a rotary table, a top drive, etc.) by rotating the entire drillstring and where drilling fluid is utilized to rotate the bit of the drillstring. In such an example, a surface RPM (SRPM) may be determined by use of the surface equipment and a downhole RPM of the mud motor may be determined using various factors related to flow of drilling fluid, mud motor type, etc. As an example, in the combined rotating mode, bit RPM can be determined or estimated as a sum of the SRPM and the mud motor RPM, assuming the SRPM and the mud motor RPM are in the same direction.

A PDM mud motor may be operated in various modes such as, for example, a rotating mode and a so-called sliding mode, which can be without rotation of a drillstring from the surface. In such an example, a bit RPM can be determined or estimated based on the RPM of the mud motor.

A RSS can drill directionally where there is continuous rotation from surface equipment, which can alleviate the sliding of a steerable motor (e.g., a PDM). A RSS may be deployed when drilling directionally (e.g., deviated, horizontal, or extended-reach wells). A RSS can aim to minimize interaction with a borehole wall, which can help to preserve borehole quality. A RSS can aim to exert a relatively consistent side force akin to stabilizers that rotate with the drillstring or orient the bit in the desired direction while continuously rotating at the same number of rotations per minute as the drillstring.

The LWD module 354 may be housed in a suitable type of drill collar and can contain one or a plurality of selected types of logging tools. It will also be understood that more than one LWD and/or MWD module can be employed, for example, as represented at by the module 356 of the drillstring assembly 350. Where the position of an LWD module is mentioned, as an example, it may refer to a module at the position of the LWD module 354, the module 356, etc. An LWD module can include capabilities for measuring, processing, and storing information, as well as for communicating with the surface equipment. In the illustrated example, the LWD module 354 may include a seismic measuring device.

The MWD module 356 may be housed in a suitable type of drill collar and can contain one or more devices for measuring characteristics of the drillstring 325 and the drill bit 326. As an example, the MWD tool 356 may include equipment for generating electrical power, for example, to power various components of the drillstring 325. As an example, the MWD tool 356 may include the telemetry equipment 352, for example, where the turbine impeller can generate power by flow of the mud; it being understood that other power and/or battery systems may be employed for purposes of powering various components. As an example, the MWD module 356 may include one or more of the following types of measuring devices: a weight-on-bit measuring device, a torque measuring device, a vibration measuring device, a shock measuring device, a stick slip measuring device, a direction measuring device, and an inclination measuring device.

FIG. 3 also shows some examples of types of holes that may be drilled. For example, consider a slant hole 372, an S-shaped hole 374, a deep inclined hole 376 and a horizontal hole 378.

A drilling operation can include directional drilling where, for example, at least a portion of a well includes a curved axis. For example, consider a radius that defines curvature where an inclination with regard to the vertical may vary until reaching an angle between about 30 degrees and about 60 degrees or, for example, an angle to about 90 degrees or possibly greater than about 90 degrees.

A directional well can include several shapes where each of the shapes may aim to meet particular operational demands. As an example, a drilling process may be performed on the basis of information as and when it is relayed to a drilling engineer. As an example, inclination and/or direction may be modified based on information received during a drilling process.

As explained, a system may be a steerable system and may include equipment to perform a method such as geosteering. A steerable system can include equipment on a lower part of a drillstring which, just above a drill bit, a bent sub may be mounted. Above directional drilling equipment, a drillstring can include MWD equipment that provides real time or near real time data of interest (e.g., inclination, direction, pressure, temperature, real weight on the drill bit, torque stress, etc.) and/or LWD equipment. As to the latter, LWD equipment can make it possible to send to the surface various types of data of interest, including for example, geological data (e.g., gamma ray log, resistivity, density and sonic logs, etc.).

The coupling of sensors providing information on the course of a well trajectory, in real time or near real time, with, for example, one or more logs characterizing the formations from a geological viewpoint, can allow for implementing a geosteering method. Such a method can include navigating a subsurface environment to follow a desired route to reach a desired target or targets.

A drillstring may include an azimuthal density neutron (ADN) tool for measuring density and porosity; a MWD tool for measuring inclination, azimuth and shocks; a compensated dual resistivity (CDR) tool for measuring resistivity and gamma ray related phenomena; one or more variable gauge stabilizers; one or more bend joints; and a geosteering tool, which may include a motor and optionally equipment for measuring and/or responding to one or more of inclination, resistivity and gamma ray related phenomena.

Geosteering can include intentional directional control of a wellbore based on results of downhole geological logging measurements in a manner that aims to keep a directional wellbore within a desired region, zone (e.g., a pay zone), etc. Geosteering may include directing a wellbore to keep the wellbore in a particular section of a reservoir, for example, to minimize gas and/or water breakthrough and, for example, to maximize economic production from a well that includes the wellbore.

Referring again to FIG. 3, the wellsite system 300 can include one or more sensors 364 that are operatively coupled to the control and/or data acquisition system 362. As an example, a sensor or sensors may be at surface locations. As an example, a sensor or sensors may be at downhole locations. As an example, a sensor or sensors may be at one or more remote locations that are not within a distance of the order of about one hundred meters from the wellsite system 300.

The system 300 can include one or more sensors 366 that can sense and/or transmit signals to a fluid conduit such as a drilling fluid conduit (e.g., a drilling mud conduit). For example, in the system 300, the one or more sensors 366 can be operatively coupled to portions of the standpipe 308 through which mud flows. As an example, a downhole tool can generate pulses that can travel through the mud and be sensed by one or more of the one or more sensors 366. In such an example, the downhole tool can include associated circuitry such as, for example, encoding circuitry that can encode signals, for example, to reduce demands as to transmission. Circuitry at the surface may include decoding circuitry to decode encoded information transmitted at least in part via mud-pulse telemetry. Circuitry at the surface may include encoder circuitry and/or decoder circuitry and circuitry downhole may include encoder circuitry and/or decoder circuitry. As an example, the system 300 can include a transmitter that can generate signals that can be transmitted downhole via mud (e.g., drilling fluid) as a transmission medium.

During drilling operations, one or more portions of a drillstring may become stuck. The term stuck can refer to one or more of varying degrees of inability to move or remove a drillstring from a bore. As an example, in a stuck condition, it might be possible to rotate pipe or lower it back into a bore or, for example, in a stuck condition, there may be an inability to move the drillstring axially in the bore, though some amount of rotation may be possible. As an example, in a stuck condition, there may be an inability to move at least a portion of the drillstring axially and rotationally.

As to the term “stuck pipe”, this can refer to a portion of a drillstring that cannot be rotated or moved axially. A condition referred to as “differential sticking” can be a condition whereby the drillstring cannot be moved (e.g., rotated or reciprocated) along the axis of the bore. Differential sticking may occur when high-contact forces caused by low reservoir pressures, high wellbore pressures, or both, are exerted over a sufficiently large area of the drillstring. Differential sticking can have time and financial cost.

A sticking force can be a product of the differential pressure between the wellbore and the reservoir and the area that the differential pressure is acting upon. This means that a relatively low differential pressure (delta p) applied over a large working area can be just as effective in sticking pipe as can a high differential pressure applied over a small area.

A condition referred to as “mechanical sticking” can be a condition where limiting or prevention of motion of the drillstring by a mechanism other than differential pressure sticking occurs. Mechanical sticking can be caused, for example, by one or more of junk in the hole, wellbore geometry anomalies, cement, keyseats or a buildup of cuttings and/or cavings in the annulus.

Various surface tools and various downhole tools can generate information germane to borehole condition. For example, images of a wall that forms a borehole during drilling can provide information as to layers, boundaries, formation characteristics, etc. In various instances, fractures may be detected via analysis of downhole imagery. Fractures can include natural fractures and fractures from resulting from drilling and/or other downhole operations. Information regarding fractures can be utilized when planning and/or performing downhole operations, which can include, for example, completions operations, drilling operations, etc. As to surface tools, consider one or more of non-destructive tools and destructive tools. As an example, a non-destructive tool may utilize microscopy, spectroscopy, etc., in a manner whereby a sample is preserved while a destructive tool may involve utilization of one or more of heat, chemicals, force, etc., which may destroy one or more aspects of a sample.

Various mud logging services can track drilling rates, lithology, visual hydrocarbon indicators, total combustible gas in mud and individual hydrocarbon compounds in the gas along with numerous drilling parameters. In various instances, a mud logger can monitor and evaluate a range of surface indicators to compile a record of subsurface geology, hydrocarbons encountered and drilling events.

For cuttings sample analysis, inside a logging unit, a mud logger can rinse and dry cuttings samples prior to examination under a binocular microscope. In such an approach, the mud logger can describe each sample, for example, in terms of lithology, color, grain size, shape, sorting, porosity, texture and one or more other characteristics that maybe relevant to rock type. Such information may be plotted in a lithology column of a mud log, which can display an estimate of gross lithology as a percentage of cuttings (e.g., reported in 10% increments).

Cuttings may be analyzed using one or more of various techniques. For example, a binocular microscope can be utilized to characterize structure of rocks in a non-destructive manner and a calcimeter can be utilized to check for presence of carbonates in a destructive manner. Through a combination of the microscopy and calcimetry, the nature of a rock sample can be discerned, for example, through implementation of one or more pre-established geology protocols.

As to calcimetry, to characterize a carbonate rock, acid such as HCl may be utilized that can react with carbonate in the rock to emit CO2 where the CO2 emissions may be detected and quantified. As mentioned, calcimetry is a destructive method and relies on an acid-base assay reaction that takes some amount of time and demands use of at least an acid at a wellsite.

Various techniques can depend on one or more human factors and, for example, may pose one or more human risks (e.g., exposure to acid, etc.). Where humans are involved in decision making, classification of rock can be discretionary, which can introduce some subjectivity or differences in opinions as to characteristics, classes, etc. As an example, a machine-based technique may offer increased reliability and more uniform or standardized protocols, optionally with reduced waiting time and/or reduce human risk.

As an example, rocks such as carbonate rocks can be characterized via diffuse reflection infrared measurement. Such a technique can be implemented in a relatively low maintenance manner to rapidly confirm a rock type and, for example, to specifically identify carbonates and clay-rich shales and discriminate limestone and dolomite rock types.

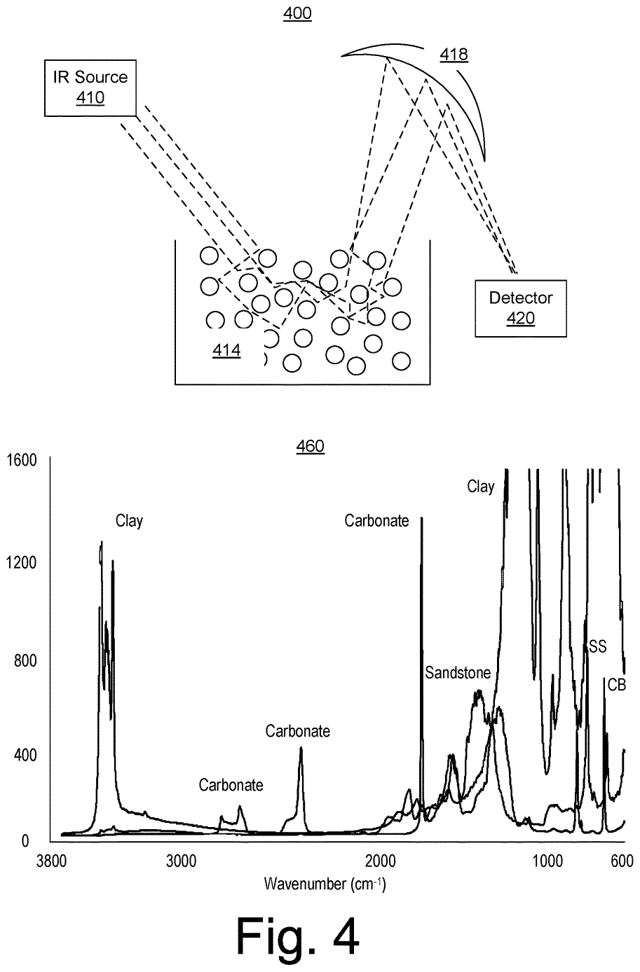

FIG. 4 shows an example of a system 400 and an example of a spectrum 460 as generated using detected diffuse reflectance. As shown, the system 400 can include an infrared (IR) source 410, a sample holder 414 with a sample, an optical element 418 (e.g., a mirror) and a detector 420. As an example, the system 400 can rely on diffuse reflectance and can be an alternative to a Diffuse Reflectance Infrared Fourier Transform Spectroscopy

(DRIFTS) system. A DRIFTS system is a type of Fourier transform infrared (FTIR) spectroscopy system that relies on diffuse reflectance as generated by irradiating a sample with infrared radiation and detecting reflected radiation that is then analyzed using a Fourier transform. FTIR spectrometers do not use gratings, but rather spectra are generated in the time domain, following the position of a moving mirror and the occurrence of constructive and destructive interference. A FTIR spectrometer relies on a processor to perform a Fast Fourier Transform (FFT) to convert a signal from a time domain to a frequency domain, which has an associated computation time. In FIG. 4, the spectrum 460 is generated using a DRIFTS system.

As shown in the example of FIG. 4, a sample can be irradiated with infrared radiation from the IR source 410 where a reflected beam of radiation is recovered and analyzed by the detector 420. The system 400 can include various components that can be robust and suitable for use in the field. For example, the system 400 can include one or more sources and/or one or more filters that provide for detection of different wavelength bands where one or more detectors may directly detect intensity signals. As to a detector or detectors, as an example, a photodetector or photodetectors may be utilized to measure intensity. Such a system may be operable without performing a Fourier transform and may have lesser maintenance and handling demands than a DRIFTS system. Such a system may be robust and portable, making it suitable for reliable use at a field site (e.g., a rigsite, etc.), for example, for analysis of cuttings as they may be generated during drilling operations.

In the example of FIG. 4, incident IR radiation can interact with a rough surface of a sample by being diffused in various directions. In such an example, the sample may be one or more cuttings that are not further diminished by grinding into a powder. As an example, a sample can be a powder sample, which may be prepared by grinding, and may be supplemented with one or more other materials. For example, a KBr powder can be utilized that can be mixed with sample powder. In such an example, the KBr powder can help to adjust (e.g., dilute) a sample where the sample may be excessively absorptive.

Signal received by a detector can depend on various factors such as, for example, surface character of a sample, reflectivity of a sample and refractive index of material in the sample. As an example, a method can utilize a system such as the system 400 to analyze mineralogy of a sample where, for example, each lithology of the sample can present identifiable and unmistakable characteristic signals such as the signals of the spectrum 460. As shown, a signal of reflectance can be recovered, and, for example, through use of a Kubelka-Munk transformation, a method can connect such a signal to a quantity dependent linearly on concentration of minerals of interest in a sample. The Kubelka-Munk transformation can be applied to a measurement such that the Kubelka-Munk transformation of the measurement is approximately proportional to the absorption coefficient and hence approximately proportional to the concentration. The Kubelka-Munk transformation may be represented as follows:

$$\frac{k}{s} = \frac{{( 1 - {R}_{∞} )}^{2}}{2 {R}_{∞}}$$

where k is the absorption coefficient of a sample as can be wavelength dependent, s is the scattering coefficient and R_{∞} is the diffuse reflectance.

As shown, the example spectrum 460 is a collection of spectra from various different materials, which include clay, carbonate (CB) and sandstone (SS). As shown, each material can be characterized by a fingerprint with peaks defined with respect to wavenumber. While the spectrum 460 is from a DRIFTS system that spans wavenumbers from 3800 cm⁻¹ to 600 cm⁻¹, as explained, a system may provide for signal detections of particular wavenumber bands (or wavelength bands) via use of one or more sources and/or one or more filters where one or more detectors may detect intensity directly (e.g., without having to perform a FFT as with a FTIR spectrometer).

As an example, an IR spectroscopic technique can include preparing a sample by grinding and/or mixing the sample with KBr to reduce effect of strong absorbing minerals such that the assumption of a function being linear with concentration is reasonable. As an example, a sample may be measured in a natural (often particulate) state where deviations from linearity at higher absorption levels may be observed. In various areas of spectroscopy, shifts away from the Kubelka-Munk equation may be observed and appropriately handled.

As an example, a system can provide for carbonate identification by using optical properties of carbonates in the IR spectrum. Such a system can operate to analyze cuttings surfaces to provide local information about one or more cuttings, with a rapid measurement that may demand no sample preparation other than drying. As an example, a system can operate to analyze a sample to differentiate between different carbonates, such as limestones and dolomites. As an example, a more accurate analysis may be provided where desired through use of one or more sample preparation techniques such as, for example, grinding a sample into a powder, which may include introduction of a material such as KBr.

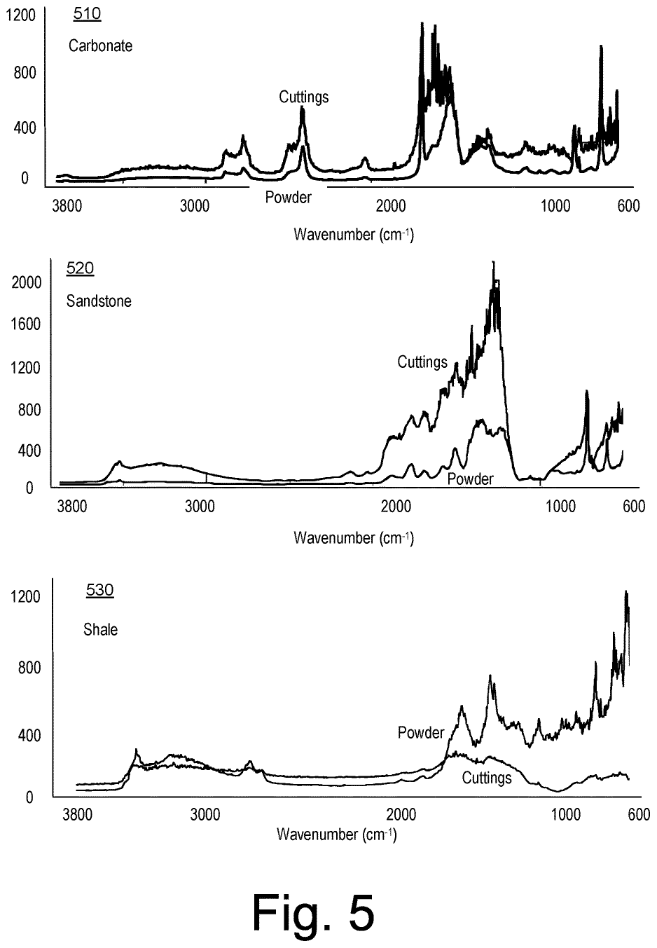

FIG. 5 shows example spectra of carbonate 510, sandstone 520 and shale 530 samples that include cuttings samples and powder samples. In the examples of FIG. 5, the spectra 510, 520 and 530 are DRIFTS system spectra on cuttings and ground cuttings (powder) to determine mineralogy. As shown in FIG. 5, the IR of non-ground cuttings can be used to clearly identify carbonate-dominated lithology using the distinctive spectral features in a wavenumber range from approximately 2000 cm⁻¹ to 3000 cm⁻¹. Various spectral features do not show an overlap with other lithologies such as, for example, sandstones or clay-rich shales.

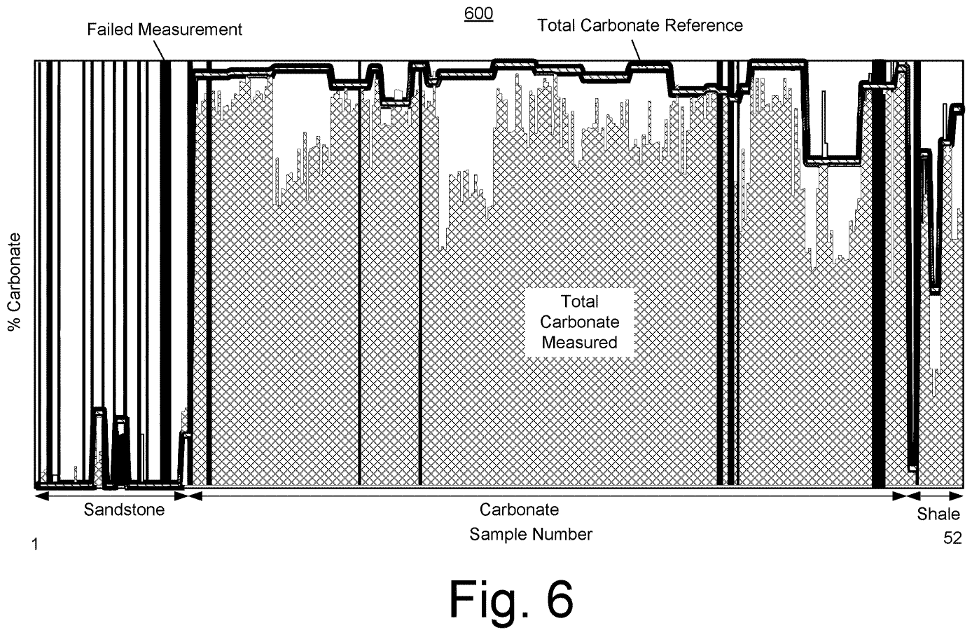

FIG. 6 shows an example plot 600 from various trials for samples including sandstone, carbonate and shale. In various trials, carbonate, non-ground cuttings yielded a diffuse reflectance IR signal of with a relatively high signal to noise ratio (SNR). The example plot 600 provides for a comparison between carbonate estimation concentrations found in non-ground cuttings with respect to a reference measured using a DRIFTS technique. In the plot 600, total carbonate measured, failed measures and total reference carbonate are shown.

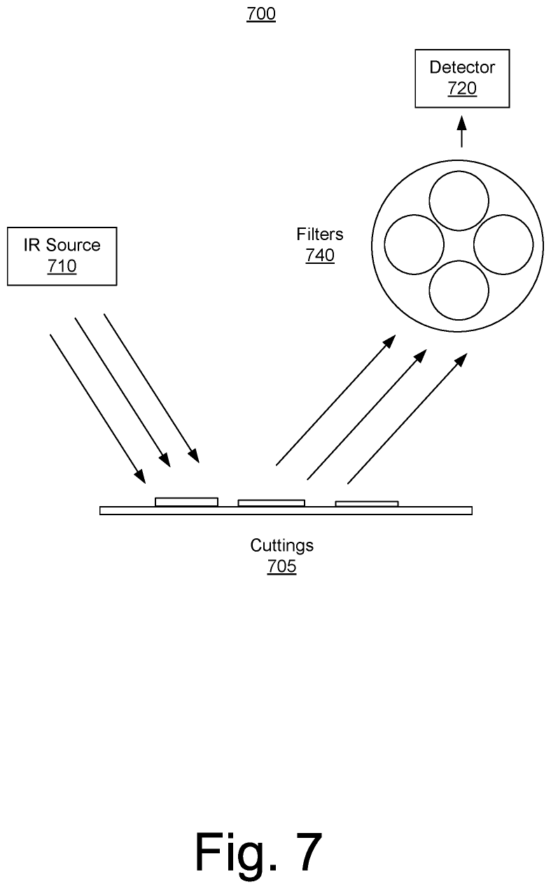

FIG. 7 shows an example of a system 700 that includes a surface as a sample holder for cuttings 705, an IR source 710, a detector 720 and filters 740, which may be arranged as individual filters, for example, on a filter wheel. The system 700 can be a diffuse reflectance system that can be an alternative to a DRIFTS system where the system 700 has an ability to focus on carbonates that can be raw dried cuttings rather than a powder, which can help to make a process more efficient and reduce human actions. As shown, the system 700 can include the detector 720 as a photodetector where a selected filter of the filters 740 may be utilized rather than a high-resolution FTIR spectrometer.

As an example, the system 700 can include one or more IR sources. For example, consider using one or more light emitting diodes (LEDs) where each of the LEDs may emit a different wavelength band. As an example, a band specific LED can include one or more features of a NANOPLUS LED (Gerbrunn, Germany). As an example, a LED can be a continuous-wave infrared LED that emits a customized wavelength band (e.g., wavenumber band). For example, consider an LED that can emit wavelengths in a band that is selected from a range of between 2800 nm and 6500 nm. As an example, an LED may be operated in a pulsed mode for reduced power consumption and/or for one or more other reasons (e.g., synchronization with one or more other LEDs, one or more detectors, one or more filters, etc.).

The system 700 can make measurements based on the optical properties of carbonates as described with respect to DRIFTS while functioning in a more streamlined manner. As an example, a system may include a broadband source emitting in the IR spectral region; an optical set-up to focus filtered light on a sample and detector; a number of filters, each with a chosen central wavelength and bandwidth; and a photodetector such as, for example, a mercury cadmium telluride (MCT), InSb, deuterated triglycine sulfate (DTGS), a pyroelectric photodetector and/or another type of photodetector. As explained, one or more band specific IR sources may be utilized, which may provide for signal detection optionally without a filter or filters disposed between a source and a detector; noting that one or more filters may be utilized with one or more band specific IR sources. As an example, a combination of IR sources may include a broadband IR source and one or more band specific IR sources.

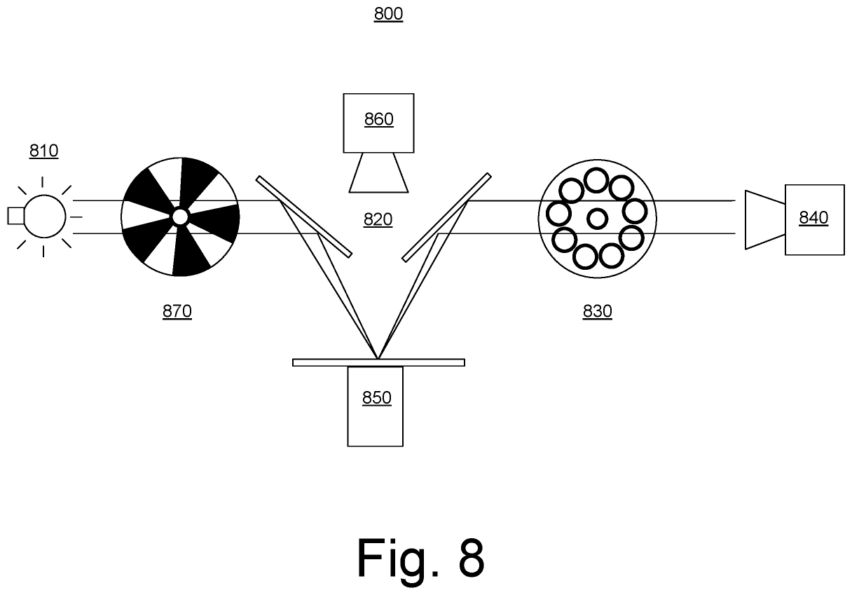

FIG. 8 shows an example of a system 800 that includes an IR source 810, a mirror assembly 820, a filter wheel 830, a detector 840, a platform 850, a camera 860 and a modulator 870 (e.g., an optical chopper). In the example of FIG. 8, the platform 850 may be movable in a direction of a z-axis to automatically position a sample with respect to a focal point of an incident beam to find a maximum signal and also movable along an x-axis and a y-axis for mapping a sample (e.g., cuttings). As an example, the camera 860 may be fixed and provide for matching each measurement with a point of a sample (e.g., a measurement point). As to the modulator 870, it may be a rotating wheel with windows and blinds that can operate as an optical chopper to modulate emissions of the IR source 810. As an example, the system 800 may include or be operatively coupled to a processor (e.g., a microcontroller, etc.), which may provide for actuating, moving, etc. For example, consider control of the sample platform 850 and the filter wheel 830 along with signal processing of signals detected by the detector 820 and images taken by the camera 860.

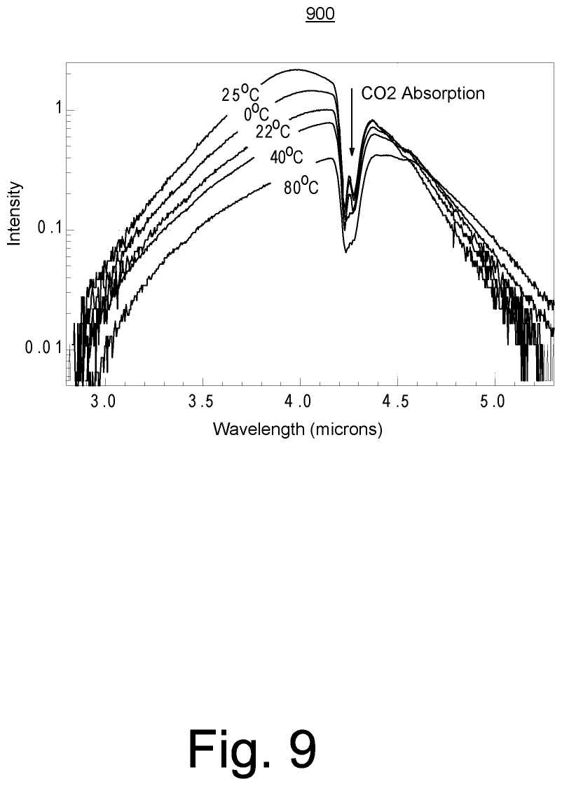

FIG. 9 shows an example plot 900 of intensity versus wavelength n microns for an IR source. As an example, an IR source can be utilized that emits a broadband beam in a range from 2000 cm⁻¹ to 3000 cm⁻¹ with relatively high output power stability. As an example, black-body radiation (e.g., a thermal light source) can be used for high temperatures (>1500 K). For example, a SiC-based heating element may be used as a broadband IR source.

In the plot 900, the data correspond to an optically immersed LED covering the spectral range 2000 cm⁻¹ to 3000 cm⁻¹. Such an LED can be controlled via its driving current and therefore can be effectively modulated electronically without using a mechanical modulator such as a mechanical chopper (e.g., as may be used to modulate a thermal IR source).

As an example, a system can include one or more optical elements. For example, one or more optical elements can be utilized to guide and/or focus a beam on a sample and/or to guide and/or focus reflected energy to one or more filters and/or one or more detectors. Optical elements may include lenses and mirrors. Optical elements can be compatible with IR radiation via use of highly transparent lenses and reflective mirrors suitable for IR radiation, for example, through use of materials such as CaF₂ for lenses and fused silica gold coating for mirrors.

As an example, an off-axis parabolic mirror or mirrors may be utilized to collect a higher solid angle of a signal and to reduce optical aberrations as may be induced by one or more IR lenses. As an example, an optic fiber or fibers may be utilized to guide a beam directly on a sample while allowing some amount of positional flexibility. As an example, a direct set-up can include one or more lamps and one or more detectors positioned in a common plane with a sample facing them, as close as practicable. Such an approach can provide for system simplicity and optionally lesser cost; though, with possibly lower resolution due to stray light.

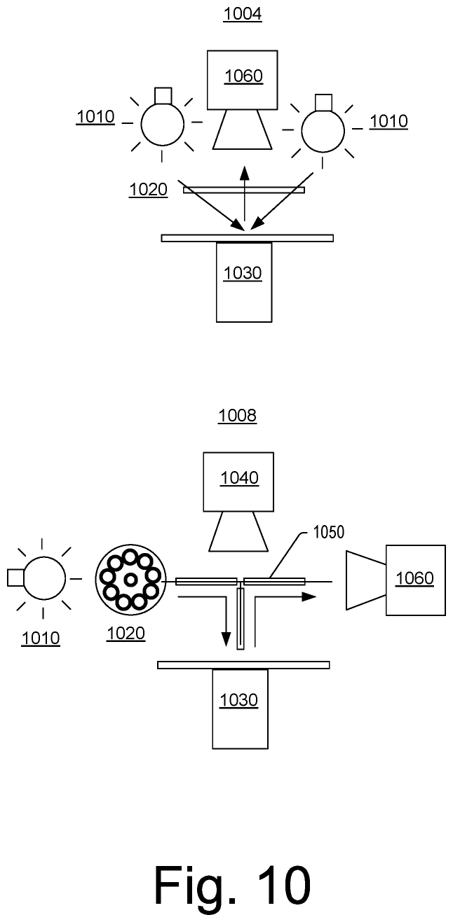

FIG. 10 shows example system arrangements of example systems 1004 and 1008 that include one or more IR sources 1010, a filter 1020, a platform 1030 and a detector 1060 and optionally a camera 1040 and optionally optic fiber 1050. In the planar arrangement of the system 1004, the number of components can be fewer than in the non-planar arrangement of the system 1008 with respect to the platform 1030.

As explained, a system can include a broadband IR source (e.g., optionally modulated using a chopper or current modulation for one or more LED IR emissions and possibly with a mounted lens to focus the light on the sample); a number of filters (e.g., a filter wheel, filters mounted directly on a multi-channel infrared detector, etc.); an adjustable platform (e.g., to adjust a sample position to match a light focus point); a camera for visible imaging (e.g., including a complementary metal-oxide semiconductor (CMOS) sensor and/or another type of sensor); an optic fiber or fibers to guide an IR signal to and/or from a sample and to photodetector; a photodetector or array of IR detectors (e.g., optionally with one or more integrated collecting lenses). As to a photodetector, one or more of mercury cadmium telluride (MCT), InSb, deuterated triglycine sulfate (DTGS) and deuterated lanthanum α-alanine doped triglycine sulfate (DLaTGS) photodetector may be utilized. As an example, a single pixel detector may be used. As an example, a MEMS spectrometer or multichannel detector with integrated filters can be used, for example, for purposes of system integration and system performance.

As an example, a detector can be selected with sufficient sensitivity. For example, consider a detector with a detectivity of 10¹⁰ cm·Hz^{1/2}·W⁻¹; noting that a detector with sensitivity lower than 109 cm·Hz^{1/2}·W⁻¹ may be sub-optimal.

As an example, one or more detectors that include a wavelength selection process may be used. Resolution of such miniaturized spectrometers tends to be relatively low (40 cm 1 to 60 cm⁻¹) compared to a standard benchtop FTIR (~4 cm⁻¹). As to some examples of miniaturized spectrometers consider one or more of the InfraTec Detector with Fabry-Pérot spectrometer (InfraTech GmbH, Dresden, Germany), the Pyreos Infrared Line Sensor Array with integrated LVF (line variable filters) (Pyreos Ltd., Edinburgh, UK), the Vigo multichannel MCT detector with 4 integrated filters (Vigo Systems, Mazowiecki, Poland) and the Si-Ware FTIR MEMS detector (Si-Ware, Paris, France).

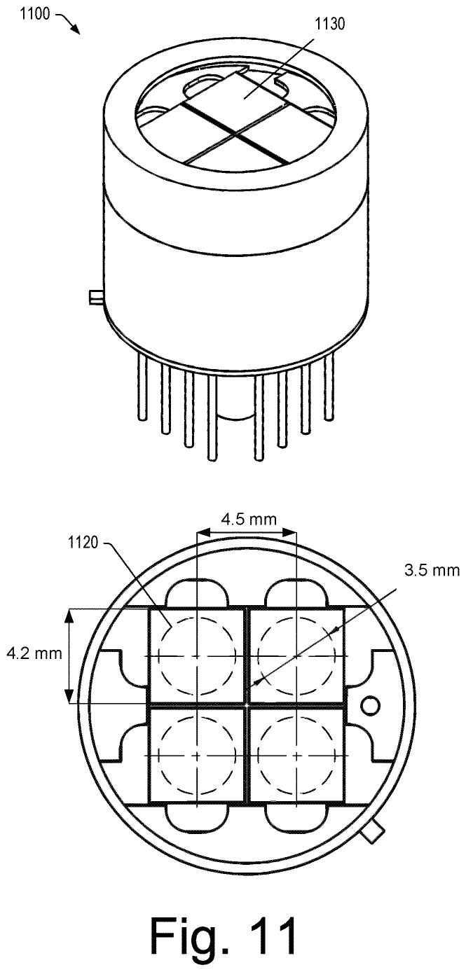

FIG. 11 shows an example of a detector 1100 that includes detector elements 1120 and filters 1130 where each of the detector elements 1120 has a corresponding one of the filters 1130. In the example of FIG. 11, the detector 1100 includes four detectors 1120 and four filters 1130, noting that fewer or more detectors and/or filters may be included. In the example of FIG. 11, detector and filter sizes and spacing are indicated. For example, a filter may be approximately 4.2 mm square and a detector aperture may be approximately 3.5 mm in diameter where a center-to-center dimension of the individual detectors is approximately 4.5 mm. Hence, the detector 1100 is relatively small and may be referred to as a micro-detector array or a micro-detector and filter array.

As to sample preparation, a system can provide for a reduction in sample preparation demands, which may facilitate use in the field and lessen human action. As explained, a system can be configured to provide for simplified sample preparation where, for example, measurement can be performed directly on dried non-ground cuttings. In various trials, measurements were acquired on dried cuttings of various shapes and sizes in a range from approximately 1 mm to approximately 3 mm. As explained, where desired, one or more additional processing actions may be taken such as, for example, grinding cuttings samples into powder and optionally introduction of a material such as KBr. Such an approach may provide for increased accuracy, if desired.

As to filters, they may be selected according to various criteria, primarily wavelength (e.g., or wavenumber). For example, a system may operate by identifying a characteristic peak of a sample that is in a range such as between approximately 2700 cm⁻¹ and approximately 2400 cm⁻¹. In such an example, the system can measure the signal in the range and compare the signal to a reference signal (e.g., background), which may be taken between 2400 cm⁻¹ and 2200 cm⁻¹ (e.g., a part of a spectrum blank for various types of rocks). As an example, one or more filters may be utilized, for example, consider one or more filters within a range from 2710 cm⁻¹ to 2650 cm⁻¹ that can allow for identification of dolomites.

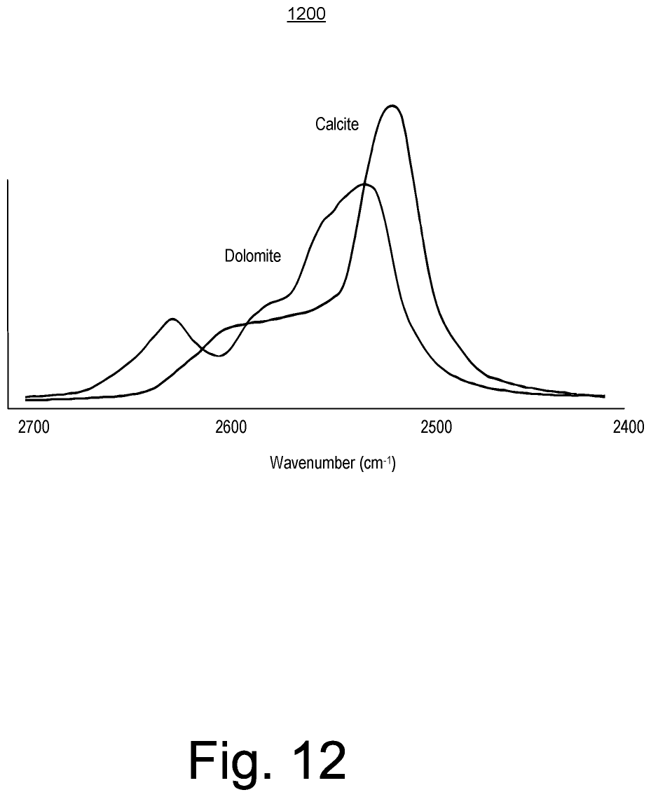

FIG. 12 shows an example of a plot 1200 for dolomite and calcite where the spectra differ such that dolomite and calcite can be distinguished. As shown, dolomite includes a peak in a range between 2700 cm⁻¹ and 2600 cm⁻¹, which may be utilized as a characteristic to identify and/or distinguish dolomite.

As to identification of carbonates, a peak at or near 2500 cm⁻¹ (e.g., plus or minus 50 cm⁻¹) can be characteristic, which may be observed for carbonate samples. Moreover, this particular wavelength corresponds to a minimum of signal for the lithologies in the plot 1200 of FIG. 12 (e.g., dolomite and calcite).

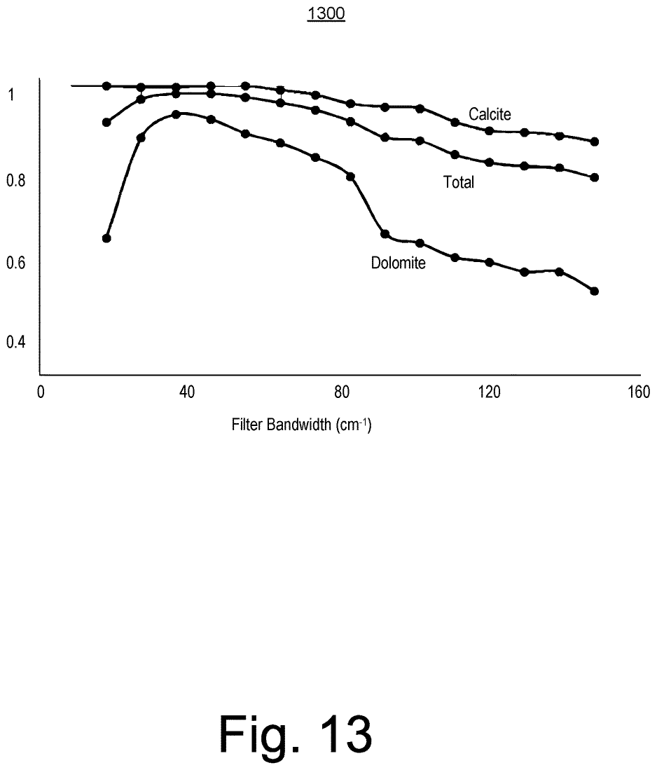

FIG. 13 shows an example plot 1300 of success rate for calcite, dolomite or total carbonates identification versus filter bandwidth spanning a filter center at 2680 cm⁻¹. However, taking the signal at a given wavelength may not always be sufficient to obtain a differentiation of lithologies. As an example, a method can include subtracting a baseline, for example, consider a baseline obtained by integrating the signal at 2300 cm⁻¹. After such a process, in various trials, a method successfully classified 97% of the spectra; where the remaining 3% were related to noisy spectra that in many instances can also be identified and discriminated.

As an example, a method can provide for differentiating between calcite and dolomite. In such an example, the method can include observing spectra related to a massif signature at approximately 2500 cm⁻¹, where, for example, a slight difference is visible between dolomite and calcite. Specifically, for dolomite a slight shift of about 10 cm⁻¹ is observed, along with a signal drop at 2600 cm⁻¹. In the example plot 1200, the spectra are measured on powder; noting that, for cuttings, the difference may be less marked, and with a variable offset. As an example, a method can include finding an optimal spectral range for cuttings that are not ground to powder. For example, consider setting two limits at 2750 cm⁻¹ and 2500 cm⁻¹, and moving them in increments of 10 cm⁻¹ to cover the set of possible combinations. In such an example, background can be removed for each increment; noting that increment size may be adjusted to be greater or lesser, which may be performed in an automated manner.

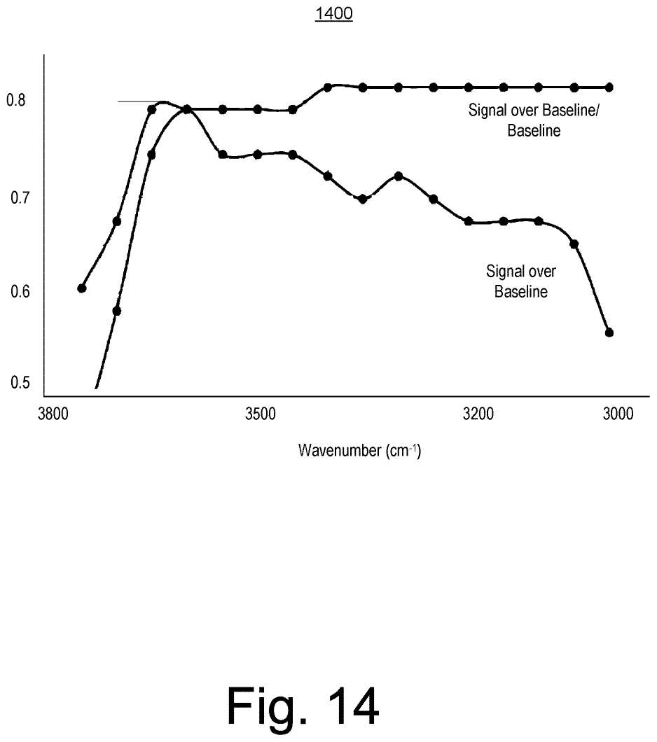

FIG. 14 shows an example plot 1400 for processed signals where one process provides absorption signal and another process provides absorption signal divided by baseline. As an example, by calculating an identification rate that could be obtained, a method can then determine an appropriate filter. For example, consider a filter with a bandwidth of 2710 cm⁻¹ to 2650 cm⁻¹. As mentioned, in various trials, 97% of the samples were successfully identified. As an example, a method can include utilizing increments and/or one or more other techniques for determining wavelength or wavenumber bands, for example, for one or more IR sources and/or one or more filters.

As an example, a method akin to a carbonates method can be implemented for samples that include clays. For example, consider the aforementioned approach as applied to target the massif at 3600 cm⁻¹. This region tends to be quite noisy, where a subtraction of noise may be insufficient to obtain acceptable results. For example, in various trials, through subtraction of noise, about 80% of the clay-rich shales were successfully identified. As an example, a method can include utilizing one or more additional filters and/or sources for better clay estimation.

As explained, a system can include filters where one or more methods can involve use of one or more of the filters. As explained, a system can include filters, a source and a detector, to identify lithologies with great precision: greater than 95% for carbonates, calcite and dolomite, and at least 80% for clays. As explained, a system may utilize one or more band specific sources, which may provide for use without one or more corresponding filters. As an example, a combination of sources and filters may be utilized.

As an example, a system can include a filter passing the interval 2400-2300 cm⁻¹ (4167-4348 nm) to measure background; a filter passing the interval 2700-2400 cm⁻¹ (3704-4167 nm) to detect carbonates; a filter passing the interval 2710-2650 cm⁻¹ (3690-3774 nm) to differentiate between calcite and dolomite; and/or a filter passing the interval 3800-3600 cm⁻¹ (2632-2778 nm) to identify clays. As an example, one or more bands may be realized by using one or more band specific sources such that, for example, a system may utilize band specific sources and/or band specific filters.

As explained, a system can include a signal modulator which may be a mechanical modulator and/or an electronic modulator (e.g., current modulation). A modulator can modulate signal amplitude. As an example, an optical chopper can be rotated and positioned as closely as possible to an IR source. As an example, an optical chopper can be combined with a filter wheel to modulate signal and wavelengths at the same time (e.g., simultaneously). As explained, an IR signal can be directly modulated by using current modulation, for example, for one or more LED light sources.

As an example, a camera can be included in a system for one or more purposes such as machine vision, which may facilitate sample tracking, mapping, positioning, etc., optionally with respect to one or more beams, optical elements, filters, modulators, etc. As explained, a camera can be positioned directly above a platform where cuttings may be positioned. In such an example, a high-resolution camera as well as a diffuse ring-LED light may be utilized for acquiring high quality images of a sample. As an example, an image may be taken with illumination from such a light and without illumination from such a light where IR radiation may be present.

As an example, a platform may be motorized akin to a 3D printer head (e.g., a 3D gantry, etc.) to allow for automated positioning of a platform with a sample based on feedback, which may be captured by a camera or cameras.

As an example, a system may be a packaged system, for example, packaged on a base, in a box, etc., which may provide for ease of carrying to a jobsite and/or protection of various components from the environment. As an example, a system may be packaged within a space of 50×20×20 cm³, which may be of lesser length. As an example, a system may include its own power source such as, for example, a battery and/or may include a socket and/or a plug for receipt of power.

As mentioned, a system can include a processor, memory, an interface, etc. As explained, a microcontroller may pilot a filter wheel, control modulation, control power to one or more IR sources, adjust a sample platform and process signals to generate results. As an example, a system can include a display or displays, which may provide for rendering of results, images taken by a camera, etc.

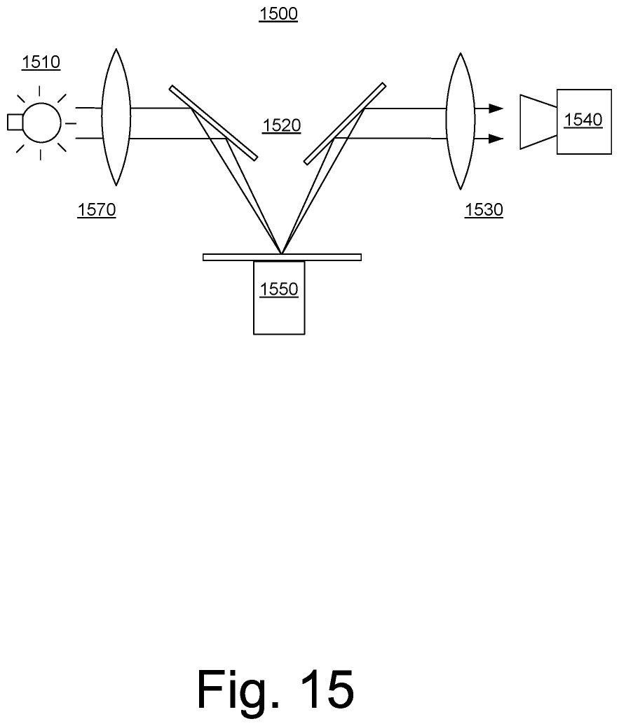

FIG. 15 shows an example of a system 1500 that includes an IR source 1510, optical elements 1520, 1530 and 1570, a platform 1550 and a detector 1540. As an example, the detector 1540 can include one or more HgCdTe (MCT) amplified photodetectors (e.g., PDAVJ5, Thorlabs, Newton, NJ). As an example, the IR source 1510 can be a stabilized tungsten IR light source (e.g., SLS202/M, Thorlabs). As an example, filters may be separate or may be integrated with the detector 1540 where the filters can include bandpass filters FB4500-500, FB4000-500 (Thorlabs), and NB-3720-095 nm (Spectrogon, Stockholm, Sweden). As an example, the optical elements 1520, 1530 and 1570 can include Ø1″ CaF₂ bi-convex lenses (e.g., LB5284-E, LB5247-E, Thorlabs) and two Ø1″ protected gold mirrors (e.g., PF10-03-M01, Thorlabs).

In various trials, a system with the following filters was utilized to perform methods for characterizing samples: CWL: 4500 nm, HW: 500 nm (FB4500-500); CWL: 4000 nm, HW: 500 nm (FB4000-500); and CWL: 3720 nm, HW: 095 nm (NB-3720-095 nm).

As mentioned, a filter wheel and an optical chopper may be included in a system. For example, consider introduction between a second lens and a detector for practical reasons. In such an example, an optical chopper frequency can be set to approximately 20 Hz. In such an example, signal from a photodetector can be visualized by an oscillator while being interpreted on a computer via a data acquisition system (DAQ) module. In such an approach, for each sample and filter, the signal can be acquired for 10 s, and a FFT performed to retrieve the signal at 20 Hz, which can be divided by the signal obtained with a perfectly reflective surface to get a reflectance rate. In various trials, tests were performed on a series of samples. In such trials, a comparison of the reflectance values from the first and second filters (e.g., at 4500 nm and 4000 nm) proved to be sufficient to highlight the presence of carbonates with an accuracy of 98% on the samples; where one of the samples was incorrectly identified due perhaps to carbonate traces being present in this sample.

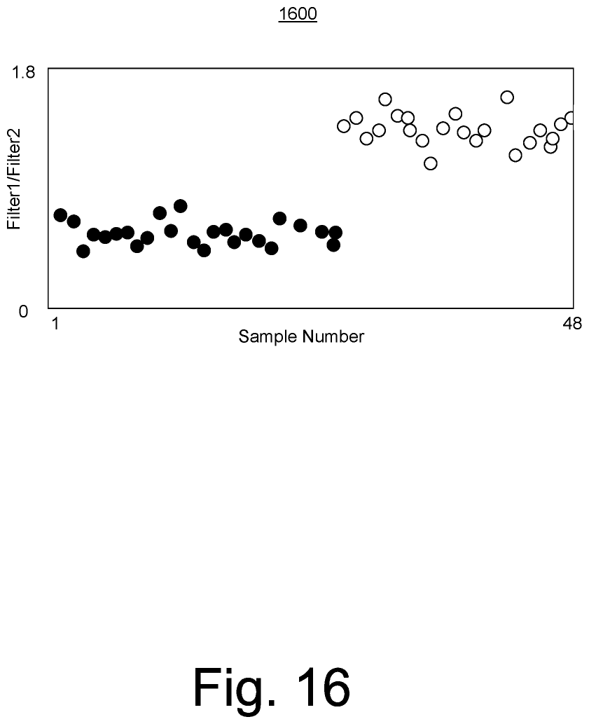

FIG. 16 shows an example plot 1600 of a ratio of filter values versus sample number. As shown, the data indicate that carbonates can be readily distinguished from non-carbonates.

As an example, a method can provide for detecting carbonates and their types in an earth sample taken from a borehole. Such a method can include using dried earth core samples, a broadband source in infrared, a photodetector working in the 3500 cm⁻¹ to 5000 cm⁻¹ range, and filters for filtering specific wavelengths. For example, such a method can include irradiating dried earth core samples using broadband IR radiation and detecting filtered reflected IR radiation as signals where processing of the signals can provide for carbonate detection and/or carbonate type detection. As explained, one or more band specific LEDs may be utilized, optionally in combination with a broadband source.

As an example, a photodetector can be a single pixel photodiode. In such an example, a single photodetector or an array may be utilized. As an example, each single pixel photodiode may have its own filter.

As an example, a photodetector and filtering wavelengths unit can be combined into a low resolution spectrometer such as a Fabry-Pérot spectrophotometer.

As an example, a system can include flat mirrors and lenses to focus and/or gather one or more IR beams (e.g., incident, reflected, etc.). As an example, one or more parabolic mirrors may be utilized to focus and/or to gather one or more IR beams (e.g., incident, reflected, etc.). As an example, one or more optic fibers may be utilized to focus and/or to gather one or more IR beams (e.g., incident, reflected, etc.).

As explained, a system may include a direct set-up configuration where a detector and an IR source are in the same plane and a sample is positioned relatively close to the detector and the IR source.

As an example, one or more characterizations from an IR analysis can be utilized in a system such as the system 200 of FIG. 2. For example, consider use in the geo data block 210, the surface models block 220, the volume models block 230, the applications block 240 (e.g., well stability assessment 246, etc.), the numerical processing block 250 and/or the operational decision block 260.

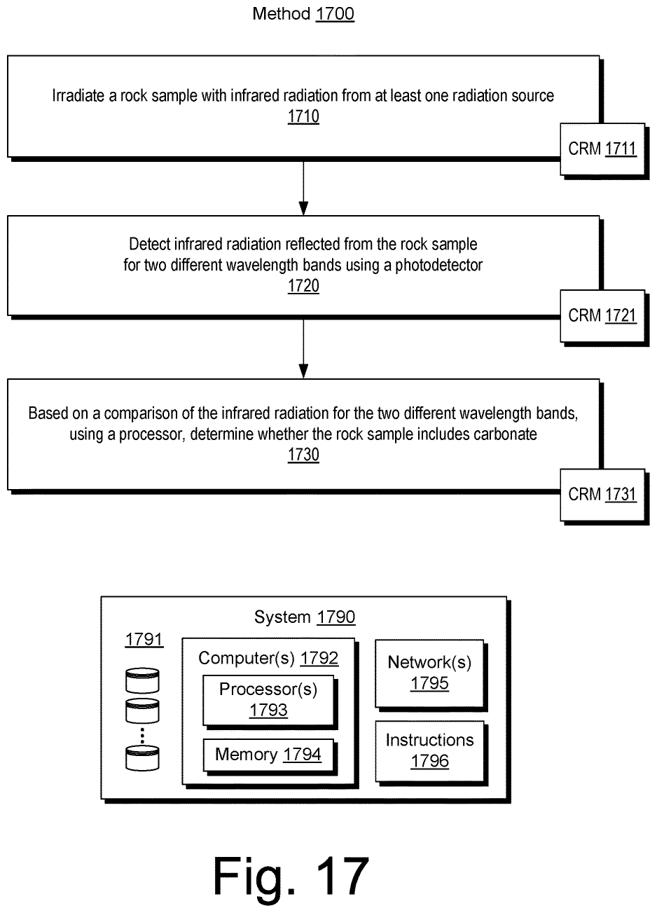

FIG. 17 shows an example of a method 1700 and an example of a system 1790. As shown, the method 1700 can include an irradiation block 1710 for irradiating a rock sample with infrared radiation from a radiation source; a detection block 1720 for detecting infrared radiation reflected from the rock sample for two different wavelength bands using a photodetector; and a determination block 1730 for, based on a comparison of the infrared radiation for the two different wavelength bands, using a processor, determining whether the rock sample includes carbonate.

The method 1700 is shown in FIG. 17 in association with various computer-readable media (CRM) blocks 1711, 1721 and 1731. Such blocks generally include instructions suitable for execution by one or more processors (or processor cores) to instruct a computing device or system to perform one or more actions. While various blocks are shown, a single medium may be configured with instructions to allow for, at least in part, performance of various actions of the method 1700. As an example, a computer-readable medium (CRM) may be a computer-readable storage medium that is non-transitory and that is not a carrier wave. As an example, one or more of the blocks 1711, 1721 and 1731 may be in the form processor-executable instructions.

In the example of FIG. 17, the system 1790 includes one or more information storage devices 1791, one or more computers 1792, one or more networks 1795 and instructions 1796. As to the one or more computers 1792, each computer may include one or more processors (e.g., or processing cores) 1793 and memory 1794 for storing the instructions 1796, for example, executable by at least one of the one or more processors 1793 (see, e.g., the blocks 1711, 1721 and 1731). As an example, a computer may include one or more network interfaces (e.g., wired or wireless), one or more graphics cards, a display interface (e.g., wired or wireless), etc.

As an example, a method can include irradiating a rock sample with infrared radiation from at least one radiation source; detecting infrared radiation reflected from the rock sample for two different wavelength bands using a photodetector; and, based on a comparison of the detected infrared radiation for the two different wavelength bands, using a processor, determining whether the rock sample includes carbonate. In such an example, the method can further include determining whether the rock sample includes calcium carbonate (calcite) and calcium magnesium carbonate (dolomite).

As an example, two different wavelength bands can be characterized by different lower limits and/or different upper limits. As an example, two different wavelength bands may overlap to some extent.

As an example, detected infrared radiation can be filtered by a first filter that passes a first wavelength band of two different wavelength bands and by a second filter that passes a second wavelength band of the two different wavelength bands.

As an example, a method can include rotating a filter wheel that includes a first filter that passes a first wavelength band of two different wavelength bands and a second filter that passes a second wavelength band of the two different wavelength bands.

As an example, a method can include irradiating a rock sample using at least one LED as a radiation source. For example, consider using a first LED for emitting infrared radiation in a first wavelength band of two different wavelength bands and a second LED for emitting infrared radiation in a second wavelength band of the two different wavelength bands. In such an example, a method may operate without filtering of reflected infrared radiation or may operate with filtering of reflected radiation.

As an example, a system for performing a method may include one or more infrared sources and/or one or more filters to provide for detection of a plurality of wavelength bands to make one or more determinations such as, for example, whether carbonates are present in a rock sample.

As an example, a photodetector can include a detector array paired with individual filters that include a first filter that passes a first wavelength band of two different wavelength bands and a second filter that passes a second wavelength band of the two different wavelength bands. As an example, a detector array or detector arrays may be configured with filters for a plurality of different wavelength bands. As an example, a detector array can include individual single pixel detectors. For example, consider an array of two, three or four single pixel detectors. In such an example, a signal may be detected from each of the single pixel detectors where a signal corresponds to a particular wavelength band. As explained, by comparing signals of different wavelength bands, a method can determine whether a rock sample includes or does not include particular material, which may be a mineral or minerals.

As an example, a method and/or a system can utilize two different wavelength bands that include a wavelength band for wavelengths including 4200 nm (2381 cm⁻¹) for background measurement and a wavelength band for wavelengths including 4000 nm (2500 cm⁻¹) for determining whether the rock sample includes carbonate.

As an example, a method can include detecting infrared radiation for more than two different wavelength bands. For example, consider a method and/or a system that utilizes a wavelength band that includes a wavelength of 4500 nm (2222 cm⁻¹), a wavelength band that includes a wavelength of 4000 nm (2500 cm⁻¹), and a wavelength band that includes a wavelength of 3720 nm (2688 cm⁻¹). As an example, consider a method and/or a system that utilizes a wavelength band for an interval between 2400-2300 cm⁻¹ (4167-4348 nm) for background measurement; a wavelength band for an interval between 2700-2400 cm⁻¹ (3704-4167 nm) for determining whether a rock sample includes carbonate; a wavelength band for an interval between 2710-2650 cm⁻¹ (3690-3774 nm) for differentiating between calcite and dolomite in the rock sample; and a wavelength band for an interval between 3800-3600 cm⁻¹ (2632-2778 nm) for determining whether the rock sample includes clay.

As an example, a method can include detecting infrared radiation reflected from a rock sample for a wavelength band to determine whether the rock sample includes clay. In such an example, the method may also include assessing one or more other wavelength bands to determine whether the rock sample includes one or more other materials (e.g., calcite, dolomite, etc.).

As an example, a method can include modulating infrared radiation by rotating an optical chopper or controlling current to an infrared source or infrared sources. As mentioned, a LED may be operable in a continuous mode or in a pulsed mode. As an example, one or more circuits that supply power to one or more LEDs may be operable in a continuous mode and/or a pulsed mode for continuous emission and/or pulsed emission, respectively.

As an example, a system can include a processor; memory accessible to the processor; and processor-executable instructions stored in the memory to instruct the system to: irradiate a rock sample with infrared radiation from at least one radiation source; detect infrared radiation reflected from the rock sample for two different wavelength bands using a photodetector; and, based on a comparison of the infrared radiation for the two different wavelength bands, determine whether the rock sample includes carbonate. In such an example, the system can include a display for rendering an image of the rock sample and for rendering one or more portions of an infrared spectrum of the rock sample.

As an example, a system can include processor-executable instructions stored in memory to instruct the system to adjust a platform for a rock sample. As an example, a system can include a camera that can capture an image of a rock sample to determine a position or positions of the rock sample. In such an example, the system may utilize one or more images for adjusting a platform and/or one or more other components of the system, for example, to properly irradiate a rock sample and to detect infrared radiation reflected from the rock sample.

As an example, one or more non-transitory computer-readable storage media can include processor-executable instructions to instruct a computing system to: irradiate a rock sample with infrared radiation from at least one radiation source; detect infrared radiation reflected from the rock sample for two different wavelength bands using a photodetector; and, based on a comparison of the infrared radiation for the two different wavelength bands, determine whether the rock sample includes carbonate. In such an example, the processor-executable instructions can be included to instruct the computing system to rotate an optical chopper to modulate the infrared radiation, to provide power to one or more LEDs, to rotate a filter wheel and/or to receive a signal from at least one photodetector, which may be a detector of a detector array. As explained, where multiple detectors are included in a system, each of the detectors may detect a different wavelength band of infrared radiation reflected from a rock sample.

As an example, a computer program product can include one or more computer-readable storage media that can include processor-executable instructions to instruct a computing system to perform one or more methods and/or one or more portions of a method.

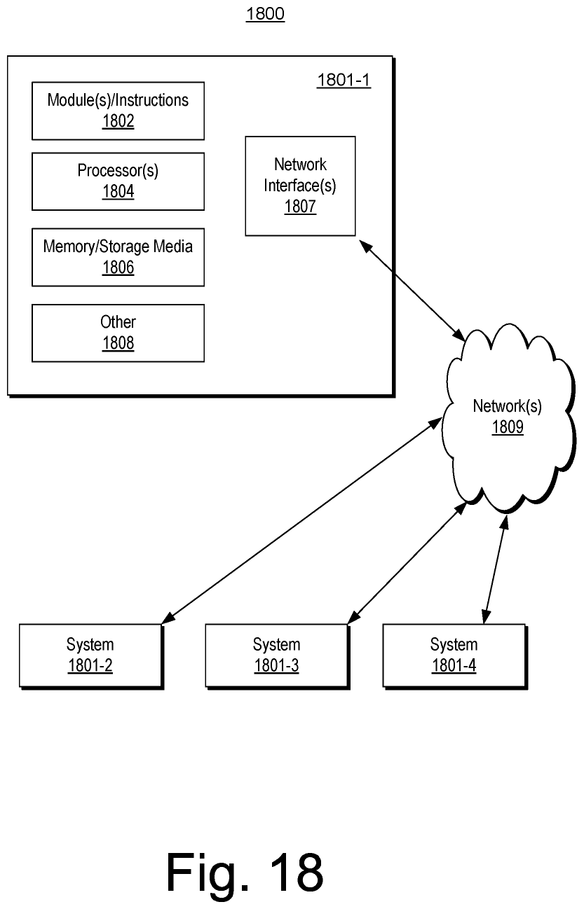

In some embodiments, a method or methods may be executed by a computing system. FIG. 18 shows an example of a system 1800 that can include one or more computing systems 1801-1, 1801-2, 1801-3 and 1801-4, which may be operatively coupled via one or more networks 1809, which may include wired and/or wireless networks. As shown, the system 1800 can include one or more other features 1808.

As an example, a system can include an individual computer system or an arrangement of distributed computer systems. In the example of FIG. 18, the computer system 1801-1 can include one or more modules 1802, which may be or include processor-executable instructions, for example, executable to perform various tasks (e.g., receiving information, requesting information, processing information, simulation, outputting information, etc.).

As an example, a module may be executed independently, or in coordination with, one or more processors 1804, which is (or are) operatively coupled to one or more storage media 1806 (e.g., via wire, wirelessly, etc.). As an example, one or more of the one or more processors 1804 can be operatively coupled to at least one of one or more network interface 1807. In such an example, the computer system 1801-1 can transmit and/or receive information, for example, via the one or more networks 1809 (e.g., consider one or more of the Internet, a private network, a cellular network, a satellite network, etc.).

As an example, the computer system 1801-1 may receive from and/or transmit information to one or more other devices, which may be or include, for example, one or more of the computer systems 1801-2, etc. A device may be located in a physical location that differs from that of the computer system 1801-1. As an example, a location may be, for example, a processing facility location, a data center location (e.g., server farm, etc.), a rig location, a wellsite location, a downhole location, etc.

As an example, a processor may be or include a microprocessor, microcontroller, processor module or subsystem, programmable integrated circuit, programmable gate array, or another control or computing device.

As an example, the storage media 1806 may be implemented as one or more computer-readable or machine-readable storage media. As an example, storage may be distributed within and/or across multiple internal and/or external enclosures of a computing system and/or additional computing systems.

As an example, a storage medium or storage media may include one or more different forms of memory including semiconductor memory devices such as dynamic or static random access memories (DRAMs or SRAMs), erasable and programmable read-only memories (EPROMs), electrically erasable and programmable read-only memories (EEPROMs) and flash memories, magnetic disks such as fixed, floppy and removable disks, other magnetic media including tape, optical media such as compact disks (CDs) or digital video disks (DVDs), BLUERAY disks, or other types of optical storage, or other types of storage devices.

As an example, a storage medium or media may be located in machine running machine-readable instructions or located at a remote site from which machine-readable instructions may be downloaded over a network for execution.

As an example, various components of a system such as, for example, a computer system, may be implemented in hardware, software, or a combination of both hardware and software (e.g., including firmware), including one or more signal processing and/or application specific integrated circuits.

As an example, a system may include a processing apparatus that may be or include a general purpose processors or application specific chips (e.g., or chipsets), such as ASICS, FPGAs, PLDs, or other appropriate devices.

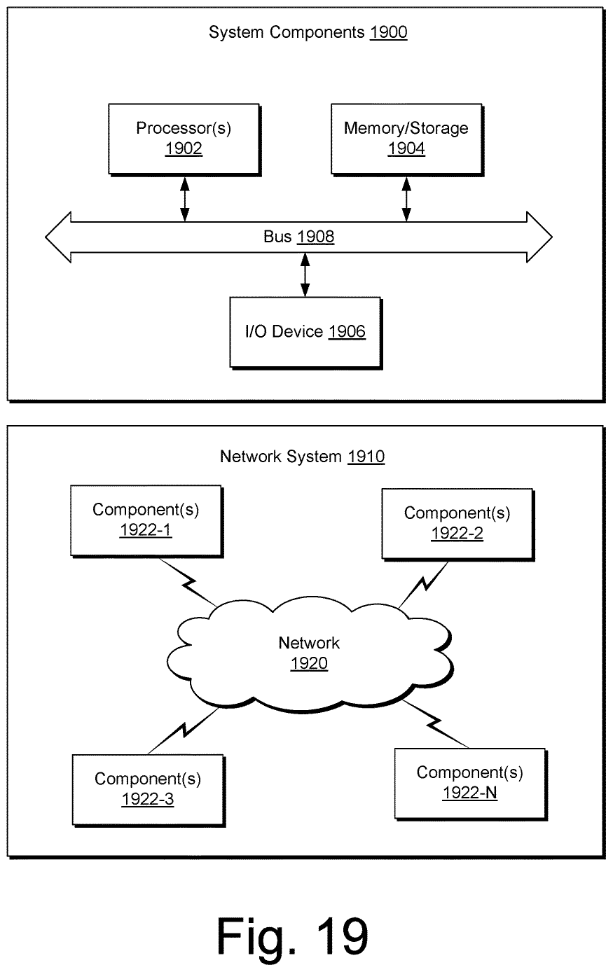

FIG. 19 shows components of an example of a computing system 1900 and an example of a networked system 1910 with a network 1920. As an example, a system for detection of carbonates can include various features of the system 1900 and/or the system 1910 of FIG. 19 and/or be operatively coupled to one or more instances of the system 1900 and/or the system 1910 of FIG. 19.

In FIG. 19, the system 1900 includes one or more processors 1902, memory and/or storage components 1904, one or more input and/or output devices 1906 and a bus 1908. In an example embodiment, instructions may be stored in one or more computer-readable media (e.g., memory/storage components 1904). Such instructions may be read by one or more processors (e.g., the processor(s) 1902) via a communication bus (e.g., the bus 1908), which may be wired or wireless. The one or more processors may execute such instructions to implement (wholly or in part) one or more attributes (e.g., as part of a method). A user may view output from and interact with a process via an I/O device (e.g., the device 1906). In an example embodiment, a computer-readable medium may be a storage component such as a physical memory storage device, for example, a chip, a chip on a package, a memory card, etc. (e.g., a computer-readable storage medium).

In an example embodiment, components may be distributed, such as in the network system 1910. The network system 1910 includes components 1922-1, 1922-2, 1922-3, . . . 1922-N. For example, the components 1922-1 may include the processor(s) 1902 while the component(s) 1922-3 may include memory accessible by the processor(s) 1902. Further, the component(s) 1922-2 may include an I/O device for display and optionally interaction with a method. The network 1920 may be or include the Internet, an intranet, a cellular network, a satellite network, etc.

As an example, a device may be a mobile device that includes one or more network interfaces for communication of information. For example, a mobile device may include a wireless network interface (e.g., operable via IEEE 802.11, ETSI GSM, BLUETOOTH, satellite, etc.). As an example, a mobile device may include components such as a main processor, memory, a display, display graphics circuitry (e.g., optionally including touch and gesture circuitry), a SIM slot, audio/video circuitry, motion processing circuitry (e.g., accelerometer, gyroscope), wireless LAN circuitry, smart card circuitry, transmitter circuitry, GPS circuitry, and a battery. As an example, a mobile device may be configured as a cell phone, a tablet, etc. As an example, a method may be implemented (e.g., wholly or in part) using a mobile device. As an example, a system may include one or more mobile devices.

As an example, a system may be a distributed environment, for example, a so-called “cloud” environment where various devices, components, etc. interact for purposes of data storage, communications, computing, etc. As an example, a device or a system may include one or more components for communication of information via one or more of the Internet (e.g., where communication occurs via one or more Internet protocols), a cellular network, a satellite network, etc. As an example, a method may be implemented in a distributed environment (e.g., wholly or in part as a cloud-based service).

As an example, information may be input from a display (e.g., consider a touchscreen), output to a display or both. As an example, information may be output to a projector, a laser device, a printer, etc. such that the information may be viewed. As an example, information may be output stereographically or holographically. As to a printer, consider a 2D or a 3D printer. As an example, a 3D printer may include one or more substances that can be output to construct a 3D object. For example, data may be provided to a 3D printer to construct a 3D representation of a subterranean formation. As an example, layers may be constructed in 3D (e.g., horizons, etc.), geobodies constructed in 3D, etc. As an example, holes, fractures, etc., may be constructed in 3D (e.g., as positive structures, as negative structures, etc.).

Although only a few example embodiments have been described in detail above, those skilled in the art will readily appreciate that many modifications are possible in the example embodiments. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures.

What is claimed is:

1. A method comprising:
irradiating a rock sample with infrared radiation from at least one radiation source;
detecting infrared radiation reflected from the rock sample for two different wavelength bands using a photodetector; and
based on a comparison of the detected infrared radiation for the two different wavelength bands, using a processor, determining whether the rock sample comprises carbonate.

2. The method of claim 1, further comprising determining whether the rock sample comprises calcium carbonate (calcite) and calcium magnesium carbonate (dolomite).

3. The method of claim 1, wherein the detected infrared radiation is filtered by a first filter that passes a first wavelength band of the two different wavelength bands and by a second filter that passes a second wavelength band of the two different wavelength bands.

4. The method of claim 1, further comprising rotating a filter wheel that comprises a first filter that passes a first wavelength band of the two different wavelength bands and a second filter that passes a second wavelength band of the two different wavelength bands.

5. The method of claim 1, wherein the at least one radiation source comprises at least one LED.

6. The method of claim 5, wherein the at least one LED comprises a first LED for emitting infrared radiation in a first wavelength band of the two different wavelength bands and a second LED for emitting infrared radiation in a second wavelength band of the two different wavelength bands.

7. The method of claim 1, wherein the photodetector comprises a detector array paired with individual filters that comprise a first filter that passes a first wavelength band of the two different wavelength bands and a second filter that passes a second wavelength band of the two different wavelength bands.

8. The method of claim 7, wherein the detector array comprises individual single pixel detectors.

9. The method of claim 1, wherein the two different wavelength bands comprise a wavelength band for wavelengths including 4200 nm (2381 cm⁻¹) for background measurement and a wavelength band for wavelengths including 4000 nm (2500 cm⁻¹) for determining whether the rock sample comprises carbonate.

10. The method of claim 1, wherein detecting infrared radiation comprises detecting infrared radiation for more wavelength bands than the two different wavelength bands.

11. The method of claim 10, wherein the more than two different wavelength bands comprise a wavelength band that includes a wavelength of 4500 nm (2222 cm⁻¹), a wavelength band that includes a wavelength of 4000 nm (2500 cm⁻¹), and a wavelength band that includes a wavelength of 3720 nm (2688 cm⁻¹).

12. The method of claim 10, wherein the more than two different wavelength bands comprise:
a wavelength band for an interval between 2400-2300 cm⁻¹ (4167-4348 nm) for background measurement;
a wavelength band for an interval between 2700-2400 cm⁻¹ (3704-4167 nm) for determining whether the rock sample comprises carbonate;
a wavelength band for an interval between 2710-2650 cm⁻¹ (3690-3774 nm) for differentiating between calcite and dolomite in the rock sample; and
a wavelength band for an interval between 3800-3600 cm⁻¹ (2632-2778 nm) for determining whether the rock sample comprises clay.

13. The method of claim 1, further comprising detecting infrared radiation reflected from the rock sample for another wavelength band to determine whether the rock sample comprises clay.

14. The method of claim 1, further comprising modulating the infrared radiation by rotating an optical chopper or controlling current to the infrared source.

15. A system comprising:
a processor;
memory accessible to the processor; and
processor-executable instructions stored in the memory to instruct the system to:
irradiate a rock sample with infrared radiation from at least one radiation source;
detect infrared radiation reflected from the rock sample for two different wavelength bands using a photodetector; and
based on a comparison of the infrared radiation for the two different wavelength bands, determine whether the rock sample comprises carbonate.

16. The system of claim 15, further comprising a display for rendering an image of the rock sample and for rendering one or more portions of an infrared spectrum of the rock sample.

17. The system of claim 15, further comprising processor-executable instructions stored in the memory to instruct the system to adjust a platform for the rock sample.

18. One or more non-transitory computer-readable storage media comprising processor-executable instructions to instruct a computing system to:
irradiate a rock sample with infrared radiation from at least one radiation source;
detect infrared radiation reflected from the rock sample for two different wavelength bands using a photodetector; and
based on a comparison of the infrared radiation for the two different wavelength bands, determine whether the rock sample comprises carbonate.

19. The one or more non-transitory computer-readable storage media of claim 18, further comprising processor-executable instructions to instruct the computing system to rotate an optical chopper to modulate the infrared radiation.

20. The one or more non-transitory computer-readable storage media of claim 18, further comprising processor-executable instructions to instruct the computing system to receive signals from a photodetector that comprises an array of detectors, wherein each of the detectors detects a different wavelength band of the infrared radiation reflected from the rock sample.
